SEQUENCE LISTING (29 sequences)

SEQ ID NO: 1 - protein, 446 aa, artificial sequence
  Heavy Chain Anti-OX40 mAb (BMS-986178)
EVQLVQSGGGLVQPGGSLRLSCAGSGFTFSSYAMYWVRQAPGKGLEWVSAIDTDAGTFYADSVRGRFTISRDNAKNSLYLQMNSLRAEDTAVYFCARLGEGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, artificial sequence
  Light Chain Anti-OX40 mAb (BMS-986178)
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 117 aa, artificial sequence
  VH Anti-OX40 mAb (BMS-986178)
EVQLVQSGGGLVQPGGSLRLSCAGSGFTFSSYAMYWVRQAPGKGLEWVSAIDTDAGTFYADSVRGRFTISRDNAKNSLYLQMNSLRAEDTAVYFCARLGEGYFFDYWGQGTLVTVSS

SEQ ID NO: 4 - DNA, 351 bp, artificial sequence
  VH Anti-OX40 mAb (BMS-986178)
gaggttcagctggtgcagtctgggggaggcttggttcagcctggggggtccctgagactctcctgtgcaggctctggattcaccttcagtagctatgctatgtactgggttcgccaggctccaggaaaaggtctggagtgggtatcagctattgatactgatgctggcacattctatgcagactccgtgcggggccgattcaccatctccagagacaatgccaagaactccttgtatcttcaaatgaacagcctgagagccgaggacacggctgtgtatttctgtgcaagacttggggaagggtacttctttgactactggggccagggaaccctggtcaccgtctcctca

SEQ ID NO: 5 - protein, 107 aa, artificial sequence
  VL Anti-OX40 mAb (BMS-986178)
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGGGTKVEIK

SEQ ID NO: 6 - DNA, 321 bp, Artificial Sequence
  VL Anti-OX40 mAb (BMS-986178)
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctcccactttcggcggagggaccaaggtggagatcaaa

SEQ ID NO: 7 - protein, 5 aa, Artificial Sequence
  Heavy Chain CDR1 Anti-OX40 mAb (BMS-986178)
SYAMY

SEQ ID NO: 8 - protein, 16 aa, artificial sequence
  Heavy Chain CDR2 Anti-OX40 mAb (BMS-986178)
AIDTDAGTFYADSVRG

SEQ ID NO: 9 - protein, 9 aa, artificial sequence
  Heavy Chain CDR3 Anti-OX40 mAb (BMS-986178)
LGEGYFFDY

SEQ ID NO: 10 - protein, 11 aa, artificial sequence
  Light Chain CDR1 Anti-OX40 mAb (BMS-986178)
RASQSVSSYLA

SEQ ID NO: 11 - protein, 7 aa, artificial sequence
  Light Chain CDR2 Anti-OX40 mAb (BMS-986178)
DASNRAT

SEQ ID NO: 12 - protein, 9 aa, artificial sequence
  Light Chain CDR3 Anti-OX40 mAb (BMS-986178)
QQRSNWPPT

SEQ ID NO: 13 - protein, 277 aa, artificial sequence
  Human OX40 Precursor Amino Acid Sequence
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 14 - protein, 189 aa, artificial sequence
  Extracellular Domain of Mature Human OX40 Amino Acid Sequence
LHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAA

SEQ ID NO: 15 - protein, 17 aa, artificial sequence
  Human OX40 Epitope
DVVSSKPCKPCTWCNLR

SEQ ID NO: 16 - protein, 329 aa, artificial sequence
  Human IgG1 constant domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 440 aa, artificial sequence
  Heavy Chain Anti-PD-1 mAb
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 214 aa, artificial sequence
  Light Chain Anti-PD-1 mAb
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 113 aa, artificial sequence [sequence omitted]

SEQ ID NO: 20 - DNA, 339 bp, Artificial Sequence
  VH Anti-PD-1 mAb
caggtgcagctggtggagtctgggggaggcgtggtccagcctgggaggtccctgagactcgactgtaaagcgtctggaatcaccttcagtaactctggcatgcactgggtccgccaggctccaggcaaggggctggagtgggtggcagttatttggtatgatggaagtaaaagatactatgcagactccgtgaagggccgattcaccatctccagagacaattccaagaacacgctgtttctgcaaatgaacagcctgagagccgaggacacggctgtgtattactgtgcgacaaacgacgactactggggccagggaaccctggtcaccgtctcctca

SEQ ID NO: 21 - protein, 107 aa, artificial sequence [sequence omitted]

SEQ ID NO: 22 - DNA, 321 bp, Artificial Sequence
  VL Anti-PD-1 mAb
gaaattgtgttgacacagtctccagccaccctgtctttgtctccaggggaaagagccaccctctcctgcagggccagtcagagtgttagtagttacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagagtagcaactggcctcggacgttcggccaagggaccaaggtggaaatcaaa

SEQ ID NO: 23 - protein, 5 aa, artificial sequence
  Heavy Chain CDR1 Anti-PD-1 mAb
NSGMH

SEQ ID NO: 24 - protein, 17 aa, artificial sequence
  Heavy Chain CDR2 Anti-PD-1 mAb
VIWYDGSKRYYADSVKG

SEQ ID NO: 25 - protein, 4 aa, artificial sequence
  Heavy Chain CDR3 Anti-PD-1 mAb
NDDY

SEQ ID NO: 26 - protein, 11 aa, artificial sequence
  Light Chain CDR1 Anti-PD-1 mAb
RASQSVSSYLA

SEQ ID NO: 27 - protein, 7 aa, artificial sequence
  Light Chain CDR2 Anti-PD-1 mAb
DASNRAT

SEQ ID NO: 28 - protein, 9 aa, artificial sequence
  Light Chain CDR3 Anti-PD-1 mAb
QQSSNWPRT

SEQ ID NO: 29 - protein, 288 aa, artificial sequence
  Complete PD-1 sequence
MQIPQAPWPVVWAVLQLGWRPGWFLDSPDRPWNPPTFFPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

(19) United States
(12) Patent Application Publication
(10) Pub. No.: US 2020/0405806 A1
(43) Pub. Date: Dec. 31, 2020

(54) COMBINATION OF A TETANUS TOXOID, ANTI-OX40 ANTIBODY AND/OR ANTI-PD-1 ANTIBODY TO TREAT TUMORS

(71) Applicant: Bristol-Myers Squibb Company, Princeton, NJ (US)

(72) Inventors: Michael QUIGLEY, Ambler, PA (US); Praveen AANUR, West Windsor, NJ (US); Zheng YANG, Plainsboro, NJ (US)

(73) Assignee: Bristol-Myers Squibb Company, Princeton, NJ (US)

(21) Appl. No.: 16/968,507

(22) PCT Filed: Feb. 7, 2019
(86) PCT No.: PCT/US2019/016963

§ 371 Date: Aug. 7, 2020

Related U.S. Application Data
(60) Provisional application No. 62/628,189, filed on Feb. 8, 2018.

(51) Int. Cl. A61K 38/16; A61P 35/00; C07K 16/28; A61K 9/00; A61K 39/00

(52) U.S. Cl. CPC A61K 38/164; A61P 35/00; C07K 16/2878; A61K 2039/545; A61K 9/0019; A61K 39/0016; C07K 16/2818

(57) ABSTRACT

Provided are methods for clinical treatment of cancers or tumors (e.g., advanced solid tumors) using (i) a combination of a tetanus toxoid, anti-OX40 antibody and anti-PD-1 antibody, (ii) a combination of anti-OX40 antibody and anti-PD-1 antibody, (iii) a combination of a tetanus toxoid and anti-PD-1 antibody, or (iv) an anti-PD-1 antibody.

### CROSS-REFERENCE TO RELATED APPLICATIONS

This PCT application claims the priority benefit of U.S. Provisional Application No. 62/628,189, filed Feb. 8, 2018, which is herein incorporated by reference in its entirety.

### REFERENCE TO SEQUENCE LISTING SUBMITTED ELECTRONICALLY

The content of the electronically submitted sequence listing in ASCII text file (Name: 3338_113PC01_Sequencelisting_ST25.txt; Size: 31,180 bytes; and Date of Creation: Jan. 31, 2019) filed with the application is herein incorporated by reference in its entirety.

### BACKGROUND

Immunotherapy for cancer has become established in recent years and is now one of the most successful and important strategies for treating patients with hematological malignancies and solid tumors. Scott et al., Cancer Immun 2012, 12:14. Aside from targeting antigens that are involved in cancer cell proliferation and survival, antibodies can also function to either activate or antagonize immunological pathways that are important in cancer immune surveillance. It is now clear that an anti-cancer antigen-specific immune response is the result of a complex dynamic interplay between antigen-presenting cells, T lymphocytes, and target cancer cells. Scott et al., Nat Rev Cancer 2012, 12(4):278-87. The T-cell anti-tumor immune response is thought to be controlled by antigen-specific stimuli sensed by the T-cell receptor (TCR) and by the combined activity of both positive (co-stimulatory) and negative (co-inhibitory) T-cell surface molecules. Gao et al., Trends Immunol 2013; 34(2):90-8. Antibodies against these key receptors have been designed and evaluated in the clinic with impressive results, heralding the onset of immunotherapy as a key pillar of anti-cancer therapy. Topalian et al., J Clin Oncol 2011; 29(36):4828-4836.

OX40 (TNFRsf4) is a 50-kD, type I transmembrane glycoprotein in the TNFRsf family of co-stimulatory receptors and is predominantly expressed by T lymphocytes, natural killer T (NKT) cells, natural killer (NK) cells, and neutrophils. OX40 is not expressed by naive T-cells and is induced on CD4, CD8, and regulatory T-cells (Treg) following stimulation through the TCR. OX40 and its ligand play a crucial role in inducing and maintaining T-cell responses. Agonism of the OX40 receptor through its ligand, typically expressed by activated antigen-presenting cells (APCs), or by OX40-specific antibodies which engage OX40 through multivalent interactions, can provide this co-stimulatory signal.

Importantly, engagement of OX40 on Tregs results in inhibition of their suppressive function. This reduction in Treg effectiveness is potentially through multiple, non-mutually exclusive mechanisms. These mechanisms include reduction of interleukin (IL)-10 production, direct activation of the effector cell rendering it less susceptible to suppression by the Treg, and direct blockade of Treg generation, which has been shown to be dependent on reduction of tumor growth factor-induced Treg generation. Aspeslagh et al., Eur J Cancer 2016; 52:50-66.

Numerous mouse models with surrogate anti-mouse-OX40 antibodies have demonstrated the effectiveness of OX40 antibody therapy. Aspeslagh et al., Eur J Cancer 2016; 52:50-66. These studies highlight the activity both as monotherapy and in combination with other immuno-oncology (IO) agents. The rat anti-mouse OX40 antibody OX86 has provided the bulk of the evidence for OX40 activity in preclinical models. OX86 also suppressed tumor growth in multiple models and combined with PD-1 blockade for enhanced efficacy. Importantly, one anti-OX40 agonist antibody has demonstrated clinical efficacy in cancer patients. The mouse antibody (9B12), when administered in patients in a Phase 1 study, demonstrated tumor regressions in 12 of the 30 treated patients with proliferation and activation of T-cell subsets. Curti et al., Cancer Res 2013; 73:7189-98. To test antibody and T cell responses to reporter antigens, the patients were also administered on day 1 either KLH or a tetanus vaccine, and on day 29 the reported antigen they did not received on day 1. A significant fold-increase in Ab response was found in patients who were immunized with tetanus or KLH on the same day as anti-OX40 compared to patients immunized 28 days later.

Programmed Cell Death 1 (PD-1) is a cell surface signaling receptor that plays a critical role in the regulation of T cell activation and tolerance (Keir M E, et al., Annu Rev Immunol 2008; 26:677-704). It is a type I transmembrane protein and together with BTLA, CTLA-4, ICOS and CD28, comprise the CD28 family of T cell co-stimulatory receptors. PD-1 is primarily expressed on activated T cells, B cells, and myeloid cells (Dong H, et al., Nat Med. 1999; 5:1365-1369). It is also expressed on natural killer (NK) cells (Terme M, et al., Cancer Res 2011; 71:5393-5399). Binding of PD-1 by its ligands, PD-L1 and PD-L2, results in phosphorylation of the tyrosine residue in the proximal intracellular immune receptor tyrosine inhibitory domain, followed by recruitment of the phosphatase SHP-2, eventually resulting in down-regulation of T cell activation. One important role of PD-1 is to limit the activity of T cells in peripheral tissues at the time of an inflammatory response to infection, thus limiting the development of autoimmunity (Pardoll D M., Nat Rev Cancer 2012; 12:252-264). Evidence of this negative regulatory role comes from the finding that PD-1-deficient mice develop lupus-like autoimmune diseases including arthritis and nephritis, along with cardiomyopathy (Nishimura H, et al., Immunity, 1999; 11:141-151; and Nishimura H, et al., Science, 2001; 291:319-322). In the tumor setting, the consequence is the development of immune resistance within the tumor microenvironment. PD-1 is highly expressed on tumor-infiltrating lymphocytes, and its ligands are up-regulated on the cell surface of many different tumors (Dong H, et al., Nat Med 2002; 8:793-800). Multiple murine cancer models have demonstrated that binding of ligand to PD-1 results in immune evasion. In addition, blockade of this interaction results in anti-tumor activity (Topalian S L, et al., NEJM 2012; 366(26):2443-2454; Hamid O, et al., NEJM 2013; 369:134-144). Moreover, it has been shown that inhibition of the PD-1/PD-L1 interaction mediates potent antitumor activity in preclinical models (U.S. Pat. Nos. 8,008,449 and 7,943,743).

Patients with metastatic or refractory solid tumors have very poor prognosis (Rosenberg S A, et al., Cancer immunotherapy in Cancer: Principles & Practice of Oncology (Eds DeVita V T, Lawrence T S and Rosenberg S A) 2011; 332-344 (Lippincott Williams & Wilkins, Philadelphia Pa.)). Despite advances in multimodal therapy, increases in overall survival in this patient population have been limited. Accordingly, it is an object of the present invention to provide improved methods for treating subjects with such tumors (e.g., advanced refractory solid tumors).

### SUMMARY OF THE INVENTION

Provided herein are methods for treating cancers or tumors in a human patient, particularly solid tumors (e.g., advanced refractory solid tumors), comprising administering to the patient a tetanus toxoid in combination with (i) an anti-OX40 and anti-PD-1 antibody or (ii) an anti-PD-1 antibody, wherein the combination is administered (or is for administration) according to a particular clinical dosage regimen (i.e., at a particular dose amount and according to a specific dosing schedule). Also provided herein are methods for treating cancers or tumors in a human patient, particularly solid tumors (e.g., advanced refractory solid tumors), comprising administering to the patient a combination of an anti-OX40 and anti-PD-1 antibody, wherein the combination is administered (or is for administration) according to a particular clinical dosage regimen (i.e., at a particular dose amount and according to a specific dosing schedule). Also provided herein are methods for treating cancers or tumors in a human patient, particularly solid tumors (e.g., advanced refractory solid tumors), comprising administering to the patient an anti-PD-1 antibody, wherein the antibody is administered (or is for administration) according to a particular clinical dosage regimen (i.e., at a particular dose amount and according to a specific dosing schedule). In one embodiment, the human patient suffers from bladder, cervical, testicular, colorectal, lung, head and neck, and ovarian cancers.

Provided herein are methods of treating cancer or a solid tumor in a human patient, the method comprising (a) administering to the patient an effective amount of a tetanus toxoid, and (b) administering to the patient after step (a) an effective amount of each of (i) an anti-OX40 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and (ii) an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21.

In one embodiment, the administering of the anti-OX40 and anti-PD-1 antibodies comprises at least one administration cycle, wherein the cycle is a period of twelve weeks, wherein for each of the at least one cycles, one dose of the anti-OX40 antibody is administered at a dose of 20, 40, or 80 mg and three doses of the anti-PD-1 antibody are administered at a dose of 480 mg.

In one embodiment, the administering of the anti-OX40 and anti-PD-1 antibodies comprises at least one administration cycle, wherein the cycle is a period of twelve weeks, wherein for each of the at least one cycles, one dose of the anti-OX40 antibody is administered at a dose sufficient to achieve about 40% OX40 receptor occupancy and three doses of the anti-PD-1 antibody are administered at a dose of 480 mg.

In one embodiment, the anti-OX40 antibody and anti-PD-1 antibody are administered at the following doses: (a) 20 mg anti-OX40 antibody and 480 mg of anti-PD-1 antibody; (b) 40 mg anti-OX40 antibody and 480 mg of anti-PD-1 antibody; or (c) 80 mg anti-OX40 antibody and 480 mg of anti-PD-1 antibody.

In one embodiment, the anti-PD-1 and anti-OX40 antibodies are formulated for intravenous administration.

In one embodiment, the anti-PD-1 and anti-OX40 antibodies are formulated together. In one embodiment, the anti-PD-1 and anti-OX40 antibodies are formulated separately.

In one embodiment, the anti-OX40 antibody is administered prior to administration of the anti-PD-1 antibody. In one embodiment, the anti-OX40 antibody is administered within about 30 minutes prior to administration of the anti-PD-1 antibody. In one embodiment, the anti-OX40 antibody is administered after administration of the anti-PD-1 antibody. In one embodiment, the anti-OX40 antibody is administered concurrently with the anti-PD-1 antibody. In one embodiment, the treatment consists of up to 9 cycles.

In one embodiment, the tetanus toxoid is administered on Day 1 of the first cycle. In one embodiment, the anti-OX40 antibody is administered on Day 1 of each cycle. In one embodiment, the anti-PD-1 antibody is administered on Days 1, 29, and 57 of each cycle.

In one embodiment, the treatment produces at least one therapeutic effect chosen from a reduction in size of a tumor, reduction in number of metastatic lesions over time, complete response, partial response, and stable disease.

In one embodiment, the cancer or solid tumor is chosen from bladder, cervical, renal cell, testicular, colorectal, lung, head and neck, and ovarian cancers. In one embodiment, the cancer or solid tumor is bladder cancer.

In one embodiment, administering to the patient an effective amount of a tetanus toxoid comprises the administration of a booster dose of the tetanus toxoid. In one embodiment, administering to the patient an effective amount of a tetanus toxoid comprises the administration of a vaccine. In one embodiment, the vaccine is Tdap, Td, DT, DTap, or an equivalent thereof. In one embodiment, the vaccine is Tdap or Td.

In one embodiment, the anti-OX40 antibody comprises (a) a heavy chain variable region CDR1 comprising the sequence set forth in SEQ ID NO:7; (b) a heavy chain variable region CDR2 comprising the sequence set forth in SEQ ID NO:8; (c) a heavy chain variable region CDR3 comprising the sequence set forth in SEQ ID NO:9; (d) a light chain variable region CDR1 comprising the sequence set forth in SEQ ID NO:10; (e) a light chain variable region CDR2 comprising the sequence set forth in SEQ ID NO:11; and (f) a light chain variable region CDR3 comprising the sequence set forth in SEQ ID NO:12. In one embodiment, the anti-OX40 antibody comprises heavy and light chain variable regions comprising the sequences set forth in SEQ ID NOs:3 and 5, respectively. In one embodiment, the anti-OX40 antibody comprises heavy and light chains comprising the sequences set forth in SEQ ID NOs:1 and 2, respectively.

In one embodiment, the anti-PD-1 antibody comprises (a) a heavy chain variable region CDR1 comprising the sequence set forth in SEQ ID NO:23; (b) a heavy chain variable region CDR2 comprising the sequence set forth in SEQ ID NO:24; (c) a heavy chain variable region CDR3 comprising the sequence set forth in SEQ ID NO:25; (d) a light chain variable region CDR1 comprising the sequence set forth in SEQ ID NO:26; (e) a light chain variable region CDR2 comprising the sequence set forth in SEQ ID NO:27; and (f) a light chain variable region CDR3 comprising the sequence set forth in SEQ ID NO:28. In one embodiment, the anti-PD-1 antibody comprises heavy and light chain variable regions comprising the sequences set forth in SEQ ID NOs:19 and 21, respectively. In one embodiment, the anti-PD-1 antibody comprises heavy and light chains comprising the sequences as set forth in SEQ ID NOs:17 and 18, respectively.

Provided herein are kits for treating a cancer or solid tumor in a human patient, the kit comprising (a) a dose of an anti-OX40 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5; (b) a dose of an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21; and (c) instructions for using the anti-OX40 antibody and anti-PD-1 antibody in any one of the methods described herein.

Provided herein is an anti-OX40 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, for co-administration to a subject in need thereof with an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21, in at least one cycle, wherein for each cycle one dose of the anti-OX40 antibody is administered at a dose of 20, 40, or 80 mg and three doses of the anti-PD-1 antibody are administered at a dose of 480 mg, and wherein an effective amount of a tetanus toxoid is administered before the administration of the anti-OX40 and anti-PD-1 antibodies.

Provided herein are methods of treating cancer or a solid tumor in a human patient, the method comprising administering to the patient an effective amount of each of (a) an anti-OX40 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and (b) an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21, and wherein the method comprises at least one administration cycle, wherein the cycle is a period of twelve weeks, wherein for each of the at least one cycles, one dose of the anti-OX40 antibody is administered at a dose of 20, 40, or 80 mg and three doses of the anti-PD-1 antibody are administered at a dose of 480 mg.

Provided herein are methods of treating a solid tumor in a human patient, the method comprising administering to the patient an effective amount of each of (a) an anti-OX40 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and (b) an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21, and wherein the method comprises at least one administration cycle, wherein the cycle is a period of twelve weeks, wherein for each of the at least one cycles, one dose of the anti-OX40 antibody is administered at a dose sufficient to achieve about 40% OX40 receptor occupancy and three doses of the anti-PD-1 antibody are administered at a dose of 480 mg.

Provided herein are kits for treating cancer or a solid tumor in a human patient, the kit comprising (a) a dose of an anti-OX40 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5; (b) a dose of an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21; and (c) instructions for using the anti-OX40 antibody and anti-PD-1 antibody in any one of the methods described herein.

Provided herein is an anti-OX40 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, for co-administration to a subject in need thereof with an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21, in at least one cycle, wherein for each cycle one dose of the anti-OX40 antibody is administered at a dose of 20, 40, or 80 mg and three doses of the anti-PD-1 antibody are administered at a dose of 480 mg.

Provided herein are methods of treating cancer or a solid tumor in a human patient, the method comprising (a) administering to the patient an effective amount of a tetanus toxoid, and (b) administering to the patient after step (a) an effective amount of an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21. In one embodiment, administering an effective amount of an anti-PD-1 antibody comprises at least one administration cycle, wherein the cycle is a period of twelve weeks, wherein for each of the at least one cycles, three doses of the anti-PD-1 antibody are administered at a dose of 480 mg.

Provided herein are kits for treating cancer or a solid tumor in a human patient, the kit comprising (a) a dose of an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21; and (b) instructions for using the anti-PD-1 antibody in any one of the methods described herein.

Provided herein is an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21, for co-administration to a subject in need thereof with an effective amount of a tetanus toxoid, in at least one cycle, wherein for each cycle three doses of the anti-PD-1 antibody are administered at a dose of 480 mg, and wherein the tetanus toxoid is administered before the administration of the anti-PD-1 antibody.

Provided herein are methods of treating a solid tumor in a human patient, the method comprising administering to the patient an effective amount of an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21, wherein the method comprises at least one administration cycle, wherein the cycle is a period of twelve weeks, wherein for each of the at least one cycles, three doses of the anti-PD-1 antibody are administered at a dose of 480 mg.

Provided herein are kits for treating a solid tumor in a human patient, the kit comprising (a) a dose of an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21; and (b) instructions for using the anti-PD-1 antibody in any one of the methods described herein.

Provided herein is an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21, for administration to a subject in need thereof in at least one cycle, wherein for each cycle three doses of the anti-PD-1 antibody are administered at a dose of 480 mg.

In another aspect of the invention, the anti-PD-1 antibody in any of the aforementioned embodiments is replaced by, or combined with, an anti-PD-L1 or anti-PD-L2 antibody. Accordingly, the invention also features methods, compositions and kits for treating cancers or tumors in human patients using the above-described clinically effective dosages of (i) a tetanus toxoid and an anti-OX40 antibody combined with the above-described clinically effective dosages of an anti-PD-1 antibody or (ii) an anti-OX40 antibody combined with the above-described clinically effective dosages of an anti-PD-1 antibody, wherein the dosage of the PD-1 antibody is replaced with the same dosage of an anti-PD-L1 or anti-PD-L2 antibody.

### BRIEF DESCRIPTION OF THE DRAWINGS

FIG. 1. Study design schematic.

FIG. 2. Study visit schematic.

FIG. 3. A list of investigational products .

### DETAILED DESCRIPTION

### I. Definitions

As used herein, the term “subject” or “patient” is a human cancer patient (e.g., a patient having an advanced solid tumor, such as an advanced refractory solid tumor).

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. A beneficial effect can also take the form of arresting, slowing, retarding, or stabilizing of a deleterious progression of a marker of solid tumor. Effective treatment may refer to alleviation of at least one symptom of a solid tumor. Such effective treatment may, e.g., reduce patient pain, reduce the size and/or number of lesions, may reduce or prevent metastasis of a tumor, and/or may slow tumor growth.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay tumor development. In some embodiments, an effective amount is an amount sufficient to prevent or delay tumor recurrence. An effective amount can be administered in one or more administrations. The effective amount of the drug or composition may: (i) reduce the number of cancer cells; (ii) reduce tumor size; (iii) inhibit, retard, slow to some extent and may stop cancer cell infiltration into peripheral organs; (iv) inhibit (i.e., slow to some extent and may stop tumor metastasis; (v) inhibit tumor growth; (vi) prevent or delay occurrence and/or recurrence of tumor; and/or (vii) relieve to some extent one or more of the symptoms associated with the cancer. In one example, an “effective amount” is the amount of tetanus toxoid, the amount of anti-OX40 antibody, and the amount of anti-PD-1 antibody, in combination, clinically proven to affect a significant decrease in cancer or slowing of progression of cancer, such as an advanced solid tumor. As used herein, the terms “fixed dose”, “flat dose” and “flat-fixed dose” are used interchangeably and refer to a dose that is administered to a patient without regard for the weight or body surface area (BSA) of the patient. The fixed or flat dose is therefore not provided as a mg/kg dose, but rather as an absolute amount of the agent (e.g., the anti-OX40 antibody and/or anti-PD-1 antibody).

As used herein, the term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent (e.g., composition comprising a combination of a tetanus toxoid, an anti-OX40 antibody, and an anti-PD-1 antibody) to the subject with the objective of reversing, alleviating, ameliorating, inhibiting, slowing down or preventing the onset, progression, development, severity or recurrence of a symptom, complication or condition, or biochemical indicia associated with a disease.

As used herein, a “body surface area (BSA)-based dose” refers to a dose (e.g., of the anti-OX40 antibody and/or anti-PD-1 antibody) that is adjusted to the body-surface area (BSA) of the individual patient. A BSA-based dose may be provided as mg/kg body weight. Various calculations have been published to arrive at the BSA without direct measurement, the most widely used of which is the Du Bois formula (see Du Bois D, Du Bois E F (June 1916) Archives of Internal Medicine 17 (6): 863-71; and Verbraecken, J. et al. (April 2006). Metabolism—Clinical and Experimental 55 (4): 515-24). Other exemplary BSA formulas include the Mosteller formula (Mosteller R D. N Engl J Med., 1987; 317:1098), the Haycock formula (Haycock G B, et al., J Pediatr 1978, 93:62-66), the Gehan and George formula (Gehan E A, George S L, Cancer Chemother Rep 1970, 54:225-235), the Boyd formula (Current, J D (1998), The Internet Journal of Anesthesiology 2 (2); and Boyd, Edith (1935), University of Minnesota. The Institute of Child Welfare, Monograph Series, No. x. London: Oxford University Press), the Fujimoto formula (Fujimoto S, et al., Nippon Eiseigaku Zasshi 1968; 5:443-50), the Takahira formula (Fujimoto S, et al., Nippon Eiseigaku Zasshi 1968; 5:443-50), and the Schlich formula (Schlich E, et al., Ernährungs Umschau 2010; 57:178-183).

As used herein, the term “flat dose” refers to a dose that is administered to a patient without regard for the weight or body surface area (BSA) of the patient. The flat dose is therefore not provided as a mg/kg dose, but rather as an absolute amount of the agent (e.g., the anti-OX40 antibody, and/or the anti-PD-1 antibody). For example, a 60 kg person and a 100 kg person would receive the same dose of an antibody (e.g., 20, 40, or 80 mg of an anti-OX40 antibody, or 480 mg of an anti-PD1 antibody).

The term “antibody” describes polypeptides comprising at least one antibody-derived antigen binding site (e.g., VH/VL region or Fv, or CDR). Antibodies include known forms of antibodies. For example, the antibody can be a human antibody, a humanized antibody, a bispecific antibody, or a chimeric antibody. The antibody also can be a Fab, Fab′2, ScFv, SMIP, Affibody®, nanobody, or a domain antibody. The antibody also can be of any of the following isotypes: IgG1, IgG2, IgG3, IgG4, IgM, IgA1, IgA2, IgAsec, IgD, and IgE. The antibody may be a naturally occurring antibody or may be an antibody that has been altered (e.g., by mutation, deletion, substitution, conjugation to a non-antibody moiety). For example, an antibody may include one or more variant amino acids (compared to a naturally occurring antibody) which changes a property (e.g., a functional property) of the antibody. For example, numerous such alterations are known in the art which affect, e.g., half-life, effector function, and/or immune responses to the antibody in a patient. The term antibody also includes artificial polypeptide constructs which comprise at least one antibody-derived antigen binding site.

An “isolated antibody” refers to an antibody that is substantially free of other antibodies having different antigenic specificities (e.g., an isolated antibody that binds specifically to OX40 or PD-1 is substantially free of antibodies that bind specifically to antigens other than OX40 or PD-1, respectively). An isolated antibody that binds specifically to OX40 can, however, have cross-reactivity to other antigens, such as OX40 molecules from different species. Moreover, an isolated antibody may be substantially free of other cellular material and/or chemicals.

The term “monoclonal antibody” (“mAb”) refers to a non-naturally occurring preparation of antibody molecules of single molecular composition, i.e., antibody molecules whose primary sequences are essentially identical, and which exhibits a single binding specificity and affinity for a particular epitope. A monoclonal antibody is an example of an isolated antibody. MAbs may be produced by hybridoma, recombinant, transgenic or other techniques known to those skilled in the art.

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the invention can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies and are used synonymously.

A “humanized antibody” refers to an antibody in which some, most or all of the amino acids outside the CDR domains of a non-human antibody are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “antigen-binding portion” of an antibody (also called an “antigen-binding fragment”) refers to one or more fragments of an antibody that retain the ability to bind specifically to the antigen bound by the whole antibody.

The term “tetanus toxoid” as used herein refers to an immunogenic compositions comprising an inactivated from of the tetanus toxin (also referred to as spasmogenic toxin, tetanus neurotoxin, or TeNT), an endopeptidase, whose toxicity has been inactivated or suppressed, for example, by chemical or heat treatment. Tetanus toxoids are described in Chapter 33 of Vaccines. (eds. Plotkin & Orenstein). 6th edition, 2012, and Farrar et al., J. Neurol. Neurosurg. Psychiatry 2000, 69, 292-301. In one embodiment, tetanus toxoid comprises a chemically (e.g., formaldehyde) inactivated tetanus toxin adsorbed to aluminum hydroxide adjuvant. Tetanus toxoid is a component of various vaccines, including, DTP, DPT, DTwP, DTaP, Tdap, DT, Td, T, and DKTP. DTaP and Tdap are combination vaccines against diphtheria, tetanus, and pertussis, wherein the upper case letters indicate higher quantity of the corresponding immunogen. Vaccines comprising a tetanus toxoid are sold in the US under various trade names, including Infanrix, DAPTACEL, Pediarix, KINRIX, Quadracel, Pentacel, ActHIB, Hiberix, TENNIVAC, Adacel, and Boostrix. Quantities of tetanus toxoid can be expressed in international units (IU) or Limit of Flocculation units (Lf).

In one embodiment, administering a tetanus toxoid to a subject comprises administering a vaccine comprising a tetanus toxoid. In one embodiment, the vaccine is the vaccine is Tdap, Td, DT, DTap, or an equivalent thereof. In one embodiment, the vaccine is Tdap or an equivalent thereof. In one embodiment, the vaccine is Td or an equivalent of Td. In one embodiment, the vaccine is DT, DTaP or and equivalent thereof. The vaccine is administered in accordance with the instructions provided in the label.

The term “OX40” as used herein refers to a receptor that is a member of the TNF-receptor superfamily, which binds to OX40 ligand (OX40-L). OX40 is also referred to as tumor necrosis factor receptor superfamily, member 4 (TNFRSF4), ACT35, IMD16, TXGP1L, and CD134. The term “OX40” includes any variants or isoforms of OX40 which are naturally expressed by cells. Accordingly, antibodies described herein may cross-react with OX40 from species other than human (e.g., cynomolgus OX40). Alternatively, the antibodies may be specific for human OX40 and may not exhibit any cross-reactivity with other species. OX40 or any variants and isoforms thereof, may either be isolated from cells or tissues which naturally express them or be recombinantly produced using well-known techniques in the art and/or those described herein.

The amino acid sequence of human OX40 precursor (Accession No. NP_003318.1) is set forth in SEQ ID NO: 13. The amino acid sequence of the extracellular domain of mature human OX40 is set forth in SEQ ID NO: 14.

As used herein, the terms “Programmed Death 1,” “Programmed Cell Death 1,” “Protein PD-1,” “PD-1,” PD1,” “PDCD1,” “hPD-1” and “hPD-I” are used interchangeably, and include variants, isoforms, species homologs of human PD-1, and analogs having at least one common epitope with PD-1. The complete PD-1 sequence can be found under GenBank Accession No. AAC51773.1 (SEQ ID NO:29) and U64863.

The protein Programmed Death 1 (PD-1) is an inhibitory member of the CD28 family of receptors, that also includes CD28, CTLA-4, ICOS and BTLA. PD-1 is expressed on activated B cells, T cells, and myeloid cells (Agata et al., supra; Okazaki et al. (2002) Curr. Opin. Immunol. 14: 391779-82; Bennett et al. (2003) J Immunol 170:711-8). The initial members of the family, CD28 and ICOS, were discovered by functional effects on augmenting T cell proliferation following the addition of monoclonal antibodies (Hutloff et al. Nature (1999); 397:263-266; Hansen et al. Immunogenics (1980); 10:247-260). PD-1 was discovered through screening for differential expression in apoptotic cells (Ishida et al. EMBO J (1992); 11:3887-95). The other members of the family, CTLA-4 and BTLA, were discovered through screening for differential expression in cytotoxic T lymphocytes and TH1 cells, respectively. CD28, ICOS and CTLA-4 all have an unpaired cysteine residue allowing for homodimerization. In contrast, PD-1 is suggested to exist as a monomer, lacking the unpaired cysteine residue characteristic in other CD28 family members.

The PD-1 gene is a 55 kDa type I transmembrane protein that is part of the Ig gene superfamily (Agata et al. (1996) Int Immunol 8:765-72). PD-1 contains a membrane proximal immunoreceptor tyrosine inhibitory motif (ITIM) and a membrane distal tyrosine-based switch motif (ITSM) (Thomas, M. L. (1995) J Exp Med 181:1953-6; Vivier, E and Daeron, M (1997) Immunol Today 18:286-91). Although structurally similar to CTLA-4, PD-1 lacks the MYPPPY motif that is critical for B7-1 and B7-2 binding. Two ligands for PD-1 have been identified, PD-L1 and PD-L2, that have been shown to downregulate T cell activation upon binding to PD-1 (Freeman et al. (2000) J Exp Med 192:1027-34; Latchman et al. (2001) Nat Immunol 2:261-8; Carter et al. (2002) Eur J Immunol 32:634-43). Both PD-L1 and PD-L2 are B7 homologs that bind to PD-1, but do not bind to other CD28 family members. PD-L1 is abundant in a variety of human cancers (Dong et al. (2002) Nat. Med. 8:787-9). The interaction between PD-1 and PD-L1 results in a decrease in tumor infiltrating lymphocytes, a decrease in T-cell receptor mediated proliferation, and immune evasion by the cancerous cells (Dong et al. (2003) J. Mol. Med. 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-L1, and the effect is additive when the interaction of PD-1 with PD-L2 is blocked as well (Iwai et al. (2002) Proc. Nat'l. Acad. Sci. USA 99:12293-7; Brown et al. (2003) J. Immunol. 170:1257-66).

Consistent with PD-1 being an inhibitory member of the CD28 family, PD-1 deficient animals develop various autoimmune phenotypes, including autoimmune cardiomyopathy and a lupus-like syndrome with arthritis and nephritis (Nishimura et al. (1999) Immunity 11:141-51; Nishimura et al. (2001) Science 291:319-22). Additionally, PD-1 has been found to play a role in autoimmune encephalomyelitis, systemic lupus erythematosus, graft-versus-host disease (GVHD), type I diabetes, and rheumatoid arthritis (Salama et al. (2003) J Exp Med 198:71-78; Prokunina and Alarcon-Riquelme (2004) Hum Mol Genet 13:R143; Nielsen et al. (2004) Lupus 13:510). In a murine B cell tumor line, the ITSM of PD-1 was shown to be essential to block BCR-mediated Ca²⁺-flux and tyrosine phosphorylation of downstream effector molecules (Okazaki et al. (2001) PNAS 98:13866-71).

“Programmed Death Ligand-1 (PD-L1)” is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and 5 analogs having at least one common epitope with hPD-L1. The complete hPD-L1 sequence can be found under GenBank Accession No. Q9NZQ7.

### IIa. Anti-OX40 Antibodies

Anti-human-OX40 antibodies (or VH/VL domains derived therefrom) suitable for use in the invention can be generated using methods well known in the art. Alternatively, art recognized anti-OX40 antibodies can be used. For example, the anti-human OX40 antibody described in U.S. Pat. No. 9,644,032, the teachings of which are hereby incorporated by reference, and referred to as monoclonal antibody OX40.21, also known as BMS-986178 can be used.

Antibodies that compete with any of the above-referenced art-recognized antibodies for binding to OX40 also can be used.

In one embodiment, the anti-OX40 antibody comprises heavy and light chains comprising the sequences shown in SEQ ID NOs:1 and 2, respectively, or antigen binding fragments and variants thereof, as described in U.S. Pat. No. 9,644,032, the teachings of which are hereby incorporated by reference. In one embodiment, the anti-OX40 antibody is BMS-986178.

In other embodiments, the antibody has the heavy and light chain CDRs or variable regions of BMS-986178. Accordingly, in one embodiment, the antibody comprises CDR1, CDR2, and CDR3 domains of the VH region of BMS-986178 having the sequence set forth in SEQ ID NO:3, and CDR1, CDR2 and CDR3 domains of the VL region of BMS-986178 having the sequence set forth in SEQ ID NO:5. In another embodiment, the antibody comprises CDR1, CDR2 and CDR3 domains comprising the sequences set forth in SEQ ID NOs:7, 8, and 9, respectively, and CDR1, CDR2 and CDR3 domains comprising the sequences set forth in SEQ ID NOs:10, 11, and 12, respectively. In another embodiment, the antibody comprises VH and/or VL regions comprising the amino acid sequences set forth in SEQ ID NO:3 and/or SEQ ID NO: 5, respectively. In another embodiment, the antibody comprises heavy chain variable (VH) and/or light chain variable (VL) regions encoded by the nucleic acid sequences set forth in SEQ ID NO:4 and/or SEQ ID NO:6, respectively. In another embodiment, the antibody competes for binding with and/or binds to the same epitope on OX40 as the above-mentioned antibodies. In another embodiment, the antibody binds an epitope of human OX40 comprising the amino acid sequence DVVSSKPCKPCTWCNLR (SEQ ID NO:15).

In another embodiment, the antibody has at least about 90% variable region amino acid sequence identity with the above-mentioned antibodies (e.g., at least about 90%, 95% or 99% variable region identity with SEQ ID NO:3 or SEQ ID NO:5).

In one embodiment, the anti-OX40 antibody is tavolixizumab (MEDI-0562), pogalizumab (MOXR0916, RG7888), GSK3174998, ATOR-1015, MEDI-6383, MEDI-6469, BMS-986178, PF-04518600, or RG7888 (MOXR0916).

### IIb. Anti-PD-1 Antibodies

Human monoclonal antibodies (HuMAbs) that bind specifically to PD-1 with high affinity have been disclosed in U.S. Pat. Nos. 8,008,449 and 8,779,105. Other anti-PD-1 mAbs have been described in, for example, U.S. Pat. Nos. 6,808,710, 7,488,802, 8,168,757 and 8,354,509, and PCT Publication No. WO 2012/145493.

In one embodiment, the anti-PD-1 antibody is nivolumab. Nivolumab (also known as “Opdivo®”; BMS-936558; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56). In another embodiment, the anti-PD-1 antibody or fragment thereof cross-competes with nivolumab. In other embodiments, the anti-PD-1 antibody or fragment thereof binds to the same epitope as nivolumab. In certain embodiments, the anti-PD-1 antibody has the same CDRs as nivolumab.

In one embodiment, the anti-PD-1 antibody comprises heavy and light chains comprising the sequences shown in SEQ ID NOs:17 and 18, respectively, or antigen binding fragments and variants thereof.

In other embodiments, the anti-PD-1 antibody has heavy and light chain CDRs or variable regions of nivolumab. Accordingly, in one embodiment, the antibody comprises CDR1, CDR2, and CDR3 domains of the VH having the sequence set forth in SEQ ID NO:19, and CDR1, CDR2 and CDR3 domains of the VL having the sequence set forth in SEQ ID NO:21. In another embodiment, the antibody comprises CDR1, CDR2 and CDR3 domains comprising the sequences set forth in SEQ ID NOs:23, 24, and 25, respectively, and CDR1, CDR2 and CDR3 domains comprising the sequences set forth in SEQ ID NOs:26, 27, and 28, respectively. In another embodiment, the antibody comprises VH and/or VL regions comprising the amino acid sequences set forth in SEQ ID NO: 19 and/or SEQ ID NO: 21, respectively. In another embodiment, the antibody comprises heavy chain variable (VH) and/or light chain variable (VL) regions encoded by the nucleic acid sequences set forth in SEQ ID NO:20 and/or SEQ ID NO:22, respectively. In another embodiment, the antibody competes for binding with and/or binds to the same epitope on PD-1 as the above-mentioned antibodies. In another embodiment, the antibody has at least about 90% variable region amino acid sequence identity with the above-mentioned antibodies (e.g., at least about 90%, 95% or 99% variable region identity with SEQ ID NO:19 or SEQ ID NO:21).

Anti-human-PD-1 antibodies (or VH and/or VL domains derived therefrom) suitable for use in the invention can be generated using methods well known in the art. Alternatively, art recognized anti-PD-1 antibodies can be used. For example, monoclonal antibodies 5C4, 17D8, 2D3, 4H1, 4A11, 7D3, and 5F4, described in WO 2006/121168, the teachings of which are hereby incorporated by reference, can be used. Other known PD-1 antibodies include Lambrolizumab (MK-3475) described in WO 2008/156712, and AMP-514 described in WO 2012/145493, the teachings of which are hereby incorporated by reference. Further known PD-1 antibodies and other PD-1 inhibitors include those described in WO 2009/014708, WO 03/099196, WO 2009/114335 and WO 2011/161699, the teachings of which are hereby incorporated by reference. Antibodies that compete with any of these art-recognized antibodies or inhibitors for binding to PD-1 also can be used.

In another embodiment, the anti-PD-1 antibody or antigen binding fragment thereof cross-competes with pembrolizumab. In some embodiments, the anti-PD-1 antibody or antigen binding fragment thereof binds to the same epitope as pembrolizumab. In certain embodiments, the anti-PD-1 antibody or antigen binding fragment thereof has the same CDRs as pembrolizumab. In another embodiment, the anti-PD-1 antibody is pembrolizumab. Pembrolizumab (also known as “Keytruda®”, lambrolizumab, and MK-3475) is a humanized monoclonal IgG4 antibody directed against human cell surface receptor PD-1 (programmed death-1 or programmed cell death-1). Pembrolizumab is described, for example, in U.S. Pat. Nos. 8,354,509 and 8,900,587. Pembrolizumab has been approved by the FDA for the treatment of relapsed or refractory melanoma.

In other embodiments, the anti-PD-1 antibody or antigen binding fragment thereof cross-competes with MEDI0608. In still other embodiments, the anti-PD-1 antibody or antigen binding fragment thereof binds to the same epitope as MEDI0608. In certain embodiments, the anti-PD-1 antibody has the same CDRs as MEDI0608. In other embodiments, the anti-PD-1 antibody is MEDI0608 (formerly AMP-514), which is a monoclonal antibody. MEDI0608 is described, for example, in U.S. Pat. No. 8,609,089.

In other embodiments, the anti-PD-1 antibody or antigen binding fragment thereof cross-competes with BGB-A317. In some embodiments, the anti-PD-1 antibody or antigen binding fragment thereof binds the same epitope as BGB-A317. In certain embodiments, the anti-PD-1 antibody or antigen binding fragment thereof has the same CDRs as BGB-A317. In certain embodiments, the anti-PD-1 antibody or antigen binding fragment thereof is BGB-A317, which is a humanized monoclonal antibody. BGB-A317 is described in U.S. Publ. No. 2015/0079109.

Anti-PD-1 antibodies suitable for use in the disclosed compositions are antibodies that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole antibodies in inhibiting ligand binding and upregulating the immune system. In certain embodiments, the anti-PD-1 antibody or antigen-binding portion thereof cross-competes with nivolumab for binding to human PD-1. In other embodiments, the anti-PD-1 antibody or antigen-binding portion thereof is a chimeric, humanized or human monoclonal antibody or a portion thereof. In certain embodiments, the antibody is a humanized antibody. In other embodiments, the antibody is a human antibody. Antibodies of an IgG1, IgG2, IgG3 or IgG4 isotype can be used.

In certain embodiments, the anti-PD-1 antibody or antigen binding fragment thereof comprises a heavy chain constant region which is of a human IgG1 or IgG4 isotype. In certain other embodiments, the sequence of the IgG4 heavy chain constant region of the anti-PD-1 antibody or antigen binding fragment thereof contains an S228P mutation which replaces a serine residue in the hinge region with the proline residue normally found at the corresponding position in IgG1 isotype antibodies. This mutation, which is present in nivolumab, prevents Fab arm exchange with endogenous IgG4 antibodies, while retaining the low affinity for activating Fc receptors associated with wild-type IgG4 antibodies (Wang et al., 2014). In yet other embodiments, the antibody comprises a light chain constant region which is a human kappa or lambda constant region. In other embodiments, the anti-PD-1 antibody or antigen binding fragment thereof is a mAb or an antigen-binding portion thereof. In certain embodiments of any of the therapeutic methods described herein comprising administration of an anti-PD-1 antibody, the anti-PD-1 antibody is nivolumab. In other embodiments, the anti-PD-1 antibody is pembrolizumab. In other embodiments, the anti-PD-1 antibody is chosen from the human antibodies 17D8, 2D3, 4H1, 4A11, 7D3 and 5F4 described in U.S. Pat. No. 8,008,449. In still other embodiments, the anti-PD-1 antibody is MEDI0608 (formerly AMP-514), AMP-224, or Pidilizumab (CT-011).

### IIc. Anti-PD-L1 Antibodies

Anti-human-PD-L1 antibodies (or VH and/or VL domains derived therefrom) suitable for use in the invention can be generated using methods well known in the art. Alternatively, art recognized anti-PD-L1 antibodies can be used. For example, human anti-PD-L1 antibodies disclosed in U.S. Pat. No. 7,943,743, the contents of which are hereby incorporated by reference, can be used. Such anti-PD-L1 antibodies include 3G10, 12A4 (also referred to as BMS-936559), 10A5, 5F8, 10H10, 1B12, 7H1, 11E6, 12B7, and 13G4. Other art recognized anti-PD-L1 antibodies which can be used include those described in, for example, U.S. Pat. Nos. 7,635,757 and 8,217,149, U.S. Publication No. 2009/0317368, and PCT Publication Nos. WO 2011/066389 and WO 2012/145493, the teachings of which also are hereby incorporated by reference. Other examples of an anti-PD-L1 antibody include atezolizumab (TECENTRIQ; RG7446), or durvalumab (IMFINZI; MEDI4736). Antibodies or antigen binding fragments thereof that compete with any of these art-recognized antibodies or inhibitors for binding to PD-L1 also can be used.

In certain embodiments, the anti-PD-L1 antibody is BMS-936559 (formerly 12A4 or MDX-1105) (see, e.g., U.S. Pat. No. 7,943,743; WO 2013/173223). In other embodiments, the anti-PD-L1 antibody is MPDL3280A (also known as RG7446 and atezolizumab) (see, e.g., Herbst et al. 2013 J Clin Oncol 31(suppl):3000; U.S. Pat. No. 8,217,149), MEDI4736 (Khleif, 2013, In: Proceedings from the European Cancer Congress 2013; Sep. 27-Oct. 1, 2013; Amsterdam, The Netherlands. Abstract 802), or MSB0010718C (also called Avelumab; see US 2014/0341917). In certain embodiments, antibodies that cross-compete for binding to human PD-L1 with, or bind to the same epitope region of human PD-L1 as the above-references PD-L1 antibodies are mAbs. For administration to human subjects, these cross-competing antibodies can be chimeric antibodies, or can be humanized or human antibodies. Such chimeric, humanized or human mAbs can be prepared and isolated by methods well known in the art.

### III. Pharmaceutical Compositions

Pharmaceutical compositions suitable for administration to human patients are typically formulated for parenteral administration, e.g., in a liquid carrier, or suitable for reconstitution into liquid solution or suspension for intravenous administration.

In general, such compositions typically comprise a pharmaceutically acceptable carrier. As used herein, the term “pharmaceutically acceptable” means approved by a government regulatory agency or listed in the U.S. Pharmacopeia or another generally recognized pharmacopeia for use in animals, particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil, glycerol polyethylene glycol ricinoleate, and the like. Water or aqueous solution saline and aqueous dextrose and glycerol solutions may be employed as carriers, particularly for injectable solutions (e.g., comprising an anti-OX40 or anti-PD-1 antibody). Liquid compositions for parenteral administration can be formulated for administration by injection or continuous infusion. Routes of administration by injection or infusion include intravenous, intraperitoneal, intramuscular, intrathecal and subcutaneous. In one embodiment, the anti-OX40 and/or anti-PD-1 antibodies are administered intravenously (e.g., in separate formulations or together (in the same formulation or in separate formulations)).

In one embodiment, the tetanus toxoid is formulated for intramuscular administration. In one embodiment, the tetanus toxoid is formulated as a vaccine.

### IV. Patient Populations

Provided herein are clinical methods for treating solid tumors or cancers (e.g., advanced refractory solid tumors) in human patients using a tetanus toxoid in combination with an anti-OX40 antibody and an anti-PD-1 antibody, or (ii) an anti-PD-1 antibody.

Also provided herein are clinical methods for treating solid tumors or cancers (e.g., advanced refractory solid tumors) in human patients using a combination of an anti-OX40 antibody and an anti-PD-1 antibody.

Also provided herein are clinical methods for treating solid tumors or cancers (e.g., advanced refractory solid tumors) in human patients using a tetanus toxoid in combination with an anti-PD-1 antibody.

Also provided herein are clinical methods for treating solid tumors or cancers (e.g., advanced refractory solid tumors) in human patients using an anti-PD-1 antibody.

Examples of cancers or solid tumors that may be treated using the methods of the invention, include liver cancer, bone cancer, pancreatic cancer, skin cancer, cancer of the head or neck, breast cancer, lung cancer, cutaneous or intraocular malignant melanoma, renal cancer, uterine cancer, ovarian cancer, colorectal cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, hematologic malignancies including, for example, multiple myeloma, B-cell lymphoma, Hodgkin lymphoma/primary mediastinal B-cell lymphoma, non-Hodgkin's lymphomas, acute myeloid lymphoma, chronic myelogenous leukemia, chronic lymphoid leukemia, follicular lymphoma, diffuse large B-cell lymphoma, Burkitt's lymphoma, immunoblastic large cell lymphoma, precursor B-lymphoblastic lymphoma, mantle cell lymphoma, acute lymphoblastic leukemia, mycosis fungoides, anaplastic large cell lymphoma, T-cell lymphoma, and precursor T-lymphoblastic lymphoma, and any combinations of said cancers. The present invention is also applicable to treatment of metastatic cancers.

In one embodiment, the human patient suffers from bladder cancer, cervical cancer, renal cell carcinoma, testicular cancer, colorectal cancer, lung cancer, head and neck cancer, or ovarian cancer.

In certain embodiments, the patient being treated with the methods described herein has an advanced solid tumor. For example, in one embodiment, the patient to be treated has cervical cancer. In another embodiment, the patient to be treated has colorectal (CRC) cancer. In another embodiment, the patient to be treated has bladder cancer (e.g., unresectable locally advanced or metastatic bladder cancer). In another embodiment, the patient to be treated has ovarian cancer (e.g., unresectable locally advanced or metastatic ovarian cancer).

In one embodiment, the patient being treated with the methods described herein has non-small cell lung cancer (NSCLC). In another embodiment, the patient to be treated has squamous cell carcinoma of the head and neck (SCCHN). In another embodiment, the patient to be treated has B-cell non-Hodgkin's lymphoma (B-NHL). In another embodiment, the patient to be treated has myeloma. In another embodiment, the patient has melanoma. In another rembodiment, the patient to be treated has diffuse large B-cell lymphoma (DLBCL).

In one embodiment, the human patient suffers from bladder cancer. In one embodiment, the bladder cancer is a locally advanced bladder cancer. In one embodiment, the bladder cancer is a metastatic bladder cancer. In one embodiment, the bladder cancer is a metastatic urothelial bladder cancer. In one embodiment, the bladder cancer is advanced urothelial carcinoma. In one embodiment, the human patient suffers from histologically or cytologically confirmed urothelial carcinoma (including mixed histologies of urothelial carcinoma with elements of other subtypes) of the renal pelvis, ureter, bladder, or urethra with progression or refractory disease. In one embodiment, the human patient suffers from bladder cancer and has been offered and/or have received or refused 1 prior platinum-based therapy for the treatment of metastatic or locally advanced unresectable disease. In one embodiment, the patient has not received more than 1 prior systemic therapy. In one embodiment, the human patient is immunotherapy treatment naïve (e.g., no prior therapy with experimental anti-tumor vaccines; any T-cell co-stimulation or checkpoint pathways, such as anti-PD-1, anti-PD-L1, anti-PD-L2, anti-CD137, or anti-CTLA-4 antibody, including ipilimumab; or other medicines specifically targeting T-cells). In one embodiment, the patient had received peri-operative (neo-adjuvant or adjuvant) treatment with a platinum agent. In one embodiment, the patient had received peri-operative (neo-adjuvant or adjuvant) treatment with a platinum agent in the setting of cystectomy for localized muscle invasive urothelial cancer. In one embodiment, the patient had received peri-operative (neo-adjuvant or adjuvant) treatment with a platinum agent in the setting of cystectomy for localized muscle invasive urothelial cancer in the prior 12 months. Sequential chemotherapy given as a planned sequence to optimize response will count as 1 regimen.

In one embodiment, the human patient suffers from cervical cancer. In one embodiment, the cervical cancer is unresectable, metastatic, or recurrent with documented disease progression.

In one embodiment, the human patient suffers from renal cell carcinoma. In one embodiment, the renal cell carcinoma is metastatic renal cell carcinoma. In one embodiment, the renal cell carcinoma is a renal cell carcinoma with a clear-cell component.

In one embodiment, the human patient suffers from testicular cancer,.

In one embodiment, the human patient suffers from colorectal cancer. In one embodiment, the colorectal cancer is a microsatellite instability-high (MSI-H) colorectal cancer. In one embodiment, the colorectal cancer is a microsatellite stable colorectal cancer. In one embodiment, the colorectal cancer is a mismatch repair-deficient colorectal cancer.

In one embodiment, the human patient suffers from lung cancer. In one embodiment, the human patient suffers from non-small cell lung cancer.

In one embodiment, the human patient suffers from head and neck cancer. In one embodiment, the head and neck cancer is squamous cell carcinoma.

In one embodiment, the human patient suffers from ovarian cancer. In one embodiment, the ovarian cancer is unresectable locally advanced ovarian cancer. In one embodiment, the ovarian cancer is metastatic ovarian cancer. In one embodiment, the ovarian cancer is recurrent platinum-sensitive ovarian cancer.

In one embodiment, the human patient suffers from melanoma.

In one embodiment, the human patient suffers from gastric adenocarcinoma.

In one embodiment, the human patient suffers from non-small cell lung cancer (NSCLC) or a virally-related cancer (e.g., a human papilloma virus (HPV)-related tumor) or gastric adenocarcinoma. In a particular embodiment, the HPV-related tumor is HPV+ head and neck cancer (HNC). In another particular embodiment, the gastric adenocarcinoma is associated with Epstein-Barr virus (EBV) infection.

In certain embodiments, the methods described herein are used to treat patients having a cancer that exhibited an inadequate response to a prior treatment, e.g., a prior treatment with an immuno-oncology drug, or patients having a cancer that is refractory or resistant, either intrinsically refractory or resistant (e.g., refractory to a PD-1 pathway antagonist), or a wherein the resistance or refractory state is acquired. For example, subjects who are not responsive or not sufficiently responsive to a first therapy or who see disease progression following treatment, e.g., anti-PD-1 treatment, may be treated by the methods described herein.

In certain embodiments, the methods described herein are used to treat patients who have not previously received (i.e., been treated with) an immuno-oncology agent, e.g., a PD-1 pathway antagonist.

In certain embodiments, the methods described herein are used in combination with a standard of care treatment (e.g., surgery, radiation, and chemotherapy). In other embodiments, the methods described herein are used as a maintenance therapy, e.g., a therapy that is intended to prevent the occurrence or recurrence of tumors.

In certain embodiments, the methods described herein are used with another treatment, e.g., radiation, surgery, or chemotherapy. For example, the methods described herein can be used when there is a risk that micrometastases may be present and/or in order to reduce the risk of a relapse.

Patients can be tested or selected for one or more of the above described clinical attributes prior to, during or after treatment.

### V. Combination Therapy

Combination therapies provided herein involve administration of (i) a tetanus toxoid in combination with an anti-OX40 antibody and an anti-PD-1 antibody, (ii) a tetanus toxoid in combination with an anti-PD-1 antibody, (iii) an anti-OX40 antibody in combination with an anti-PD-1 antibody, or (iv) an anti-PD-1 antibody to treat subjects having cancer or solid tumors (e.g., advanced refractory solid tumors). In one embodiment, a method provided herein comprises the administration of a tetanus toxoid, an anti-OX40 antibody and an anti-PD-1 antibody. In one embodiment, a method provided herein comprises the administration of a tetanus toxoid and an anti-PD-1 antibody. In one embodiment, a method provided herein comprises the administration of an anti-OX40 antibody and an anti-PD-1 antibody. In one embodiment, a method provided herein comprises the administration of an anti-PD-1 antibody.

In one embodiment, the invention provides a tetanus toxoid, an anti-OX40 antibody, and an anti-PD-1 antibody in combination according to a defined clinical dosage regimen, to treat subjects having cancer or a solid tumor (e.g., an advanced refractory solid tumor). In one embodiment, the invention provides a tetanus toxoid and an anti-PD-1 antibody in combination according to a defined clinical dosage regimen, to treat subjects having cancer or a solid tumor (e.g., an advanced refractory solid tumor). In a particular embodiment, the tetanus toxoid is Tdap. In another embodiment, the anti-OX40 antibody is BMS-986178. In another embodiment, the anti-PD-1 antibody is nivolumab. In another embodiment, dosage regimens are adjusted to provide the optimum desired response (e.g., an effective response).

In one embodiment, the invention provides an anti-OX40 antibody and an anti-PD-1 antibody in combination according to a defined clinical dosage regimen, to treat subjects having cancer or a solid tumor (e.g., an advanced refractory solid tumor). In one embodiment, the invention provides an anti-PD-1 antibody according to a defined clinical dosage regimen, to treat subjects having cancer or a solid tumor (e.g., an advanced refractory solid tumor). In one embodiment, the anti-OX40 antibody is BMS-986178. In one embodiment, the anti-PD-1 antibody is nivolumab. In one embodiment, dosage regimens are adjusted to provide the optimum desired response (e.g., an effective response).

As used herein, adjunctive or combined administration (coadministration) includes simultaneous administration of the compounds in the same or different dosage form, or separate administration of the compounds (e.g., sequential administration).

In one embodiment, the tetanus toxoid is administered first, followed by the simultaneous administration of the anti-OX40 and anti-PD-1 antibodies in a single formulation. In one embodiment, the anti-OX40 and anti-PD-1 antibodies can be formulated for separate administration and are administered concurrently or sequentially (e.g., one antibody is administered within about 30 minutes prior to administration of the second antibody) after the administration of the tetanus toxoid. In another embodiment the anti-OX40 antibody is administered within about 30 minutes (e.g., within about 29, 28, 27, 26, 25, 24, 23, 22, 21, 20, or less minutes) before or after the administration of the anti-PD-1 antibody.

In one embodiment, the anti- OX40 and anti-PD-1 antibodies can be simultaneously administered in a single formulation. Alternatively, the anti-OX40 and anti-PD-1 antibodies can be formulated for separate administration and are administered concurrently or sequentially (e.g., one antibody is administered within about 30 minutes plior to administration of the second antibody). In one embodiment the anti-OX40 antibody is administered within about 30 minutes (e.g., within about 29, 28, 27, 26, 25, 24, 23, 22, 21, 20, or less minutes) before or after the administration of the anti-PD-1 antibody.

In one embodiment, the anti-OX40 antibody can be administered first followed by (e.g., immediately followed by) the administration of the anti-PD-1 antibody, or vice versa. In one embodiment, the anti-OX40 antibody is administered prior to administration of the anti-PD-1 antibody. In another embodiment, the anti-OX40 antibody is administered after administration of the anti-PD-1 antibody. In another embodiment, the anti-OX40 antibody and anti-PD-1 antibody are administered concurrently. Such concurrent or sequential administration preferably results in both antibodies being simultaneously present in treated patients.

In one embodiment, the tetanus toxoid is administered first, followed by the administration of the anti-PD-1 antibody.

### VI. Treatment Protocols

In one embodiment, a method for treating cancer or a solid tumor in a human patient include administering to the patient an effective amount of each of:

(a) a tetanus toxoid;

(b) an anti-OX40 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5,

(c) an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21,

wherein the tetanus toxoid is administered before the administration of the anti-OX40 and anti-PD-1 antibodies. In one embodiment, the administering of the anti-OX40 and anti-PD-1 antibodies comprises at least one administration cycle, wherein for each of the at least one cycles, at least one dose of the anti-OX40 antibody is administered at a flat dose of about 1, 3, 10, 20, 40, 50, 80, 100, 130, 150, 160, 180, 200, 240 or 320 mg and at least one dose of the anti-PD-1 antibody is administered at flat dose of about 50, 80, 100, 120, 150, 180, 200, 240, 360, 480, 720, or 960 mg. In another embodiment, at least one dose of the anti-OX40 antibody is administered at a dose of 0.01, 0.03, 0.25, 0.1, 0.3, 1 or 3, 5, 8 or 10 mg/kg body weight and at least one dose of the anti-PD-1 antibody is administered at a dose of 0.1, 0.3, 1, 3, 5, 8 or 10 mg/kg body weight. In one embodiment, the administering of the anti-OX40 and anti-PD-1 antibodies comprises at least one administration cycle, wherein the cycle is a period of 12 weeks, wherein for each of the at least one cycles, at least one dose of the anti-OX40 antibody is administered at a flat dose of about 120, 40, or 80 mg and at least 3 doses of the anti-PD-1 antibody are administered at flat dose of about 480 mg.

In one embodiment, a method for treating cancer or a solid tumor in a human patient include administering to the patient an effective amount of each of:

(a) an anti-OX40 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5,

(b) an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21,

wherein the administering of the anti-OX40 and anti-PD-1 antibodies comprises at least one administration cycle, wherein for each of the at least one cycles, at least one dose of the anti-OX40 antibody is administered at a flat dose of about 1, 3, 10, 20, 40, 50, 80, 100, 130, 150, 160, 180, 200, 240 or 360 mg and at least one dose of the anti-PD-1 antibody is administered at flat dose of about 50, 80, 100, 120, 150, 160, 180, 200, 240, 480, 720, or 960 mg. In another embodiment, at least one dose of the anti-OX40 antibody is administered at a dose of 0.01, 0.03, 0.25, 0.1, 0.3, 1 or 3, 5, 8 or 10 mg/kg body weight and at least one dose of the anti-PD-1 antibody is administered at a dose of 0.1, 0.3, 1, 3, 5, 8 or 10 mg/kg body weight. In one embodiment, the cycle is a period of 12 weeks, wherein for each of the at least one cycles, at least one dose of the anti-OX40 antibody is administered at a flat dose of about 20, 40, or 80 mg and at least 3 doses of the anti-PD-1 antibody are administered at flat dose of about 480 mg.

In one embodiment, the anti-OX40 antibody and anti-PD-1 antibody are administered at the following doses:

(a) 20 mg anti-OX40 antibody and 240, 360, or 480 mg of anti-PD-1 antibody;

(b) 40 mg anti-OX40 antibody and 240, 360, or 480 mg of anti-PD-1 antibody;

(c) 80 mg anti-OX40 antibody and 240, 360, or 480 mg of anti-PD-1 antibody;

(d) 160 mg anti-OX40 antibody and 240, 360, or 480 mg of anti-PD-1 antibody;

(e) 320 mg anti-OX40 antibody and 240, 360, or 480 mg of anti-PD-1 antibody.

In one embodiment, the anti-OX40 antibody and anti-PD-1 antibody are administered at the following doses:

(a) 10 mg anti-OX40 antibody and 480 mg of anti-PD-1 antibody;

(b) 20 mg anti-OX40 antibody and 480 mg of anti-PD-1 antibody;

(c) 40 mg anti-OX40 antibody and 480 mg of anti-PD-1 antibody;

(d) 80 mg anti-OX40 antibody and 480 mg of anti-PD-1 antibody; or

(e) 160 mg anti-OX40 antibody and 480 mg of anti-PD-1 antibody.

In one embodiment, the anti-OX40 antibody and anti-PD-1 antibody are administered at 10 mg anti-OX40 antibody and 480 mg of anti-PD-1 antibody. In one embodiment, the anti-OX40 antibody and anti-PD-1 antibody are administered at 20 mg anti-OX40 antibody and 480 mg of anti-PD-1 antibody. In one embodiment, the anti-OX40 antibody and anti-PD-1 antibody are administered at 40 mg anti-OX40 antibody and 480 mg of anti-PD-1 antibody. In one embodiment, the anti-OX40 antibody and anti-PD-1 antibody are administered at 80 mg anti-OX40 antibody and 480 mg of anti-PD-1 antibody. In one embodiment, the anti-OX40 antibody and anti-PD-1 antibody are administered at 160 mg anti-OX40 antibody and 480 mg of anti-PD-1 antibody.

In one embodiment, the anti-OX40 antibody and anti-PD-1 antibody are administered at the following doses:

(a) 20 mg anti-OX40 antibody and 240 mg of anti-PD-1 antibody;

(b) 40 mg anti-OX40 antibody and 240 mg of anti-PD-1 antibody;

(c) 80 mg anti-OX40 antibody and 240 mg of anti-PD-1 antibody;

(d) 160 mg anti-OX40 antibody and 240 mg of anti-PD-1 antibody; or

(e) 320 mg anti-OX40 antibody and 240 mg of anti-PD-1 antibody.

In one embodiment, the anti-OX40 antibody and anti-PD-1 antibody are administered at the following doses:

(a) 20 mg anti-OX40 antibody and 360 mg of anti-PD-1 antibody;

(b) 40 mg anti-OX40 antibody and 360 mg of anti-PD-1 antibody;

(c) 80 mg anti-OX40 antibody and 360 mg of anti-PD-1 antibody;

(d) 160 mg anti-OX40 antibody and 360 mg of anti-PD-1 antibody; or

(e) 320 mg anti-OX40 antibody and 360 mg of anti-PD-1 antibody.

In one embodiment, the anti-OX40 antibody and anti-PD-1 antibody are administered at the following doses:

(a) 20 mg anti-OX40 antibody and 480 mg of anti-PD-1 antibody;

(b) 40 mg anti-OX40 antibody and 480 mg of anti-PD-1 antibody;

(c) 80 mg anti-OX40 antibody and 480 mg of anti-PD-1 antibody;

(d) 160 mg anti-OX40 antibody and 480 mg of anti-PD-1 antibody; or

(e) 320 mg anti-OX40 antibody and 480 mg of anti-PD-1 antibody.

In another embodiment, the anti-OX40 antibody and anti-PD-1 antibody are administered at the following doses:

(a) 0.3 mg/kg anti-OX40 antibody and 1 mg/kg of anti-PD-1 antibody;

(b) 0.3 mg/kg anti-OX40 antibody and 3 mg/kg of anti-PD-1 antibody;

(c) 0.25 mg/kg anti-OX40 antibody and 3 mg/kg of anti-PD-1 antibody;

(d) 1 mg/kg anti-OX40 antibody and 3 mg/kg of anti-PD-1 antibody; or

(e) 3 mg/kg anti-OX40 antibody and 3 mg/kg of anti-PD-1 antibody.

In one embodiment, a method for treating cancer or a solid tumor in a human patient include administering to the patient an effective amount of each of:

(a) a tetanus toxoid; and

(b) an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21,

wherein the tetanus toxoid is administered before the administration of the anti-PD-1 antibody. In one embodiment, the administering of the anti-PD-1 antibody comprises at least one administration cycle, wherein for each of the at least one cycles, at least one dose of the anti-PD-1 antibody is administered at flat dose of about 50, 80, 100, 120, 150, 180, 200, 240, 360, 480, 720, or 960 mg. In another embodiment, at least one dose of the anti-PD-1 antibody is administered at a dose of 0.1, 0.3, 1, 3, 5, 8 or 10 mg/kg body weight. In one embodiment, the administering of the anti-PD-1 antibody comprises at least one administration cycle, wherein the cycle is a period of 12 weeks, wherein for each of the at least one cycles, at least 3 doses of the anti-PD-1 antibody are administered at flat dose of about 480 mg.

In one embodiment, a method for treating cancer or a solid tumor in a human patient include administering to the patient an effective amount of:

an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21,

wherein the method comprises at least one administration cycle, wherein for each of the at least one cycles, at least one dose of the anti-PD-1 antibody is administered at flat dose of about 50, 80, 100, 120, 150, 180, 200, 240, 360, 480, 720, or 960 mg. In another embodiment, at least one dose of the anti-PD-1 antibody is administered at a dose of 0.1, 0.3, 1, 3, 5, 8 or 10 mg/kg body weight. In one embodiment, the cycle is a period of 12 weeks, wherein for each of the at least one cycles, at least 3 doses of the anti-PD-1 antibody are administered at flat dose of about 480 mg.

In one embodiment, the anti-PD-1 antibody is administered at a dose of 240 mg. In one embodiment, the anti-PD-1 antibody is administered at a dose of 360 mg. In one embodiment, the anti-PD-1 antibody is administered at a dose of 480 mg.

In one embodiment, the dose of the anti-OX40 and/or anti-PD-1 antibody is calculated per body weight, e.g., mg/kg body weight. In another embodiment, the dose of the anti-OX40 and/or anti-PD-1 antibody is a flat-fixed dose. In another embodiment, the dose of the anti-OX40 and/or anti-PD-1 antibody is varied over time. For example, the anti-OX40 antibody and/or anti-PD-1 antibody may be initially administered at a high dose and may be lowered over time. In another embodiment, the anti-OX40 antibody and/or anti-PD-1 antibody is initially administered at a low dose and increased over time.

In another embodiment, the amount of the anti-OX40 and/or anti-PD-1 antibodies administered is constant for each dose. In another embodiment, the amount of antibody administered varies with each dose. For example, the maintenance (or follow-on) dose of the antibody can be higher or the same as the loading dose which is first administered. In another embodiment, the maintenance dose of the antibody can be lower or the same as the loading dose.

In one embodiment, the tetanus toxoid is formulated for intramuscular administration.

In one embodiment, the anti-OX40 and/or anti-PD-1 antibodies are formulated for intravenous administration.

In one embodiment, the tetanus toxoid is administered on Day 1 of Cycle 1.

In one embodiment, the anti-PD-1 antibody is administered at least on Day 1 of each cycle. In one embodiment, the anti-OX40 antibody is administered at least on Day 1 of each cycle. In one embodiment, the anti-PD-1 antibody is administered on Days 1, 29, and 57 of each cycle. In one embodiment, the anti-OX40 antibody is administered on Day 1 of each cycle.

In one embodiment, the anti-OX40 and/or anti-PD-1 antibodies are administered once per week, once every two weeks, once every three weeks, once every four weeks, once a month, once every 3 months, once every 4 months, or once every three to 6 months. In one embodiment, the anti-OX40 antibody is administered once per week, once every two weeks, once every three weeks, once every four weeks, once a month, once every 3 months, once every 4 months, or once every three to 6 months. In one embodiment, the anti-OX40 antibody is administered once every 12 weeks, once every 16 weeks, or once every 12 to 24 weeks. In one embodiment, the anti-PD-1 antibody is administered once per week, once every two weeks, once every three weeks, once every four weeks, once a month, once every 3 months, once every 4 months, or once every three to 6 months. In one embodiment, the anti-PD-1 antibody is administered once every 12 weeks, once every 16 weeks, or once every 12 to 24 weeks. In one embodiment, the anti-OX40 and/or anti-PD-1 antibodies are administered as long as a clinical benefit is observed or until there is a complete response, confirmed progressive disease or unmanageable toxicity.

In another embodiment, a cycle of administration is 1, 2, 3, 4, 8, 12, 16, 20, or, 24 weeks, which can be repeated, as necessary. In one embodiment, a cycle of administration is 1 week. In one embodiment, a cycle of administration is 2 weeks. In one embodiment, a cycle of administration is 3 weeks. In one embodiment, a cycle of administration is 4 weeks. In one embodiment, a cycle of administration is 8 weeks. In one embodiment, a cycle of administration is 12 weeks. In one embodiment, a cycle of administration is 16 weeks. In one embodiment, a cycle of administration is 20 weeks. In one embodiment, a cycle of administration is 24 weeks. In another embodiment, the treatment consists of up to 2, 3, 4, 5, 6, 7, 8, 9, or 10 cycles. In one embodiment, the treatment consists of 2 cycles. In one embodiment, the treatment consists of 3 cycles. In one embodiment, the treatment consists of 4 cycles. In one embodiment, the treatment consists of 5 cycles. In one embodiment, the treatment consists of 6 cycles. In one embodiment, the treatment consists of 7 cycles. In one embodiment, the treatment consists of 8 cycles. In one embodiment, the treatment consists of 9 cycles. In one embodiment, the treatment consists of 10 cycles.

In another embodiment, a cycle of administration is 12 weeks and the treatment consists of up to 9 cycles.

In another embodiment, 1, 2, 3, 4, 6, or 8 doses of the anti-PD-1 antibody are administered per cycle. In one embodiment, 1 dose of the anti-PD-1 antibody is administered per cycle. In one embodiment, 2 doses of the anti-PD-1 antibody are administered per cycle. In one embodiment, 3 doses of the anti-PD-1 antibody are administered per cycle. In one embodiment, 4 doses of the anti-PD-1 antibody are administered per cycle. In another embodiment, 1, 2, 3, or 4 doses of the anti-OX40 antibody are administered per cycle. In one embodiment, 1 dose of the anti-OX40 antibody is administered per cycle. In one embodiment, 2 doses of the anti-OX40 antibody are administered per cycle. In one embodiment, 3 doses of the anti-OX40 antibody are administered per cycle. In one embodiment, 4 doses of the anti-OX40 antibody are administered per cycle.

In another embodiment, 3 doses of the anti-PD-1 antibody and 1 dose of the anti-OX40 antibody is administered per 12-week cycle.

In another embodiment, the tetanus toxoid, anti-OX40 antibody, and anti-PD-1 antibody are administered as a first line of treatment (e.g., the initial or first treatment). In another embodiment, the tetanus toxoid, anti-OX40 antibody, and anti-PD-1 antibody are administered as a second line of treatment (e.g., after the initial or first treatment, including after relapse and/or where the first treatment has failed).

In another embodiment, the anti-OX40 and anti-PD-1 antibodies are administered as a first line of treatment (e.g., the initial or first treatment). In another embodiment, the anti-OX40 and anti-PD-1 antibodies are administered as a second line of treatment (e.g., after the initial or first treatment, including after relapse and/or where the first treatment has failed).

In another embodiment, the tetanus toxoid and anti-PD-1 antibody are administered as a first line of treatment (e.g., the initial or first treatment). In another embodiment, the tetanus toxoid and anti-PD-1 antibody are administered as a second line of treatment (e.g., after the initial or first treatment, including after relapse and/or where the first treatment has failed).

In another embodiment, the anti-PD-1 antibody is administered as a first line of treatment (e.g., the initial or first treatment). In another embodiment, the anti-PD-1 antibody is administered as a second line of treatment (e.g., after the initial or first treatment, including after relapse and/or where the first treatment has failed).

Provided herein are methods for optimizing anti-OX40 dosing regimens using a tetanus recall immune response. In one embodiment, the method comprises administering to a subject an effective dose of a tetanus toxoid and an anti-OX40 antibody, and measuring the anti-tetanus recall immune response, wherein the anti-OX40 antibody is administered according to a defined dosing regimen. In one embodiment, the method further comprises comparing the recall immune response induced by the tetanus toxoid and anti-OX40 antibody to a recall immune response induced by the tetanus toxoid in the absence of the administration of the anti-OX40 antibody. In one embodiment, the method comprises comparing the anti-tetanus recall immune responses induced by the administration of an effective dose of a tetanus toxoid and different dosing regimens of the anti-OX40 antibody. In one embodiment, the method further comprises identifying an optimal dosing regimen for the anti-OX40 antibody based at least in part on the anti-tetanus recall immune responses induced by the different anti-OX40 antibody dosing regimens. In one embodiment, the method further comprises the administration of an anti-PD-1 antibody according to a defined dosing regimen.

In one embodiment, the defined anti-OX40 and/or anti-PD-1 antibody dosing regimen comprises at least one administration cycle. In one embodiment, the defined anti-OX40 and/or anti-PD-1 antibody dosing regimen comprises up to 1, 2, 3, 4, 5, 6, 7, 8, or 9 administration cycles. In one embodiment, the at least one administration cycle comprises the administration of 1, 2, 3, 4, 5, 6, or more doses of the anti-OX40 and/or anti-PD-1 antibody. In one embodiment, doses of the anti-OX40 antibody are administered once per week, once every two weeks, once every three weeks, once every four weeks, once a month, once every 3 months, once every 4 months, or once every three to 6 months. In one embodiment, doses of the anti-OX40 and/or anti-PD-1 antibody are administered on the same day of each administration cycle.

In one embodiment, the anti-OX40 antibody comprises (a) a heavy chain variable region CDR1 comprising the sequence set forth in SEQ ID NO:7; (b) a heavy chain variable region CDR2 comprising the sequence set forth in SEQ ID NO:8; (c) a heavy chain variable region CDR3 comprising the sequence set forth in SEQ ID NO:9; (d) a light chain variable region CDR1 comprising the sequence set forth in SEQ ID NO:10; (e) a light chain variable region CDR2 comprising the sequence set forth in SEQ ID NO:11; and (f) a light chain variable region CDR3 comprising the sequence set forth in SEQ ID NO:12. In one embodiment, the anti-OX40 antibody comprises heavy and light chain variable regions comprising the sequences set forth in SEQ ID NOs:3 and 5, respectively. In one embodiment, the anti-OX40 antibody comprises heavy and light chains comprising the sequences set forth in SEQ ID NOs:1 and 2, respectively. In one embodiment, the anti-OX40 antibody is BMS-986178.

In one embodiment, the anti-PD-1 antibody comprises (a) a heavy chain variable region CDR1 comprising the sequence set forth in SEQ ID NO:23; (b) a heavy chain variable region CDR2 comprising the sequence set forth in SEQ ID NO:24; (c) a heavy chain variable region CDR3 comprising the sequence set forth in SEQ ID NO:25; (d) a light chain variable region CDR1 comprising the sequence set forth in SEQ ID NO:26; (e) a light chain variable region CDR2 comprising the sequence set forth in SEQ ID NO:27; and (f) a light chain variable region CDR3 comprising the sequence set forth in SEQ ID NO:28. In one embodiment, the anti-PD-1 antibody comprises heavy and light chain variable regions comprising the sequences set forth in SEQ ID NOs:19 and 21, respectively. In one embodiment, the anti-PD-1 antibody comprises heavy and light chains comprising the sequences as set forth in SEQ ID NOs:17 and 18, respectively

In one embodiment, the measuring the anti-tetanus immune response comprises determining the level of anti-tetanus antibody in the subject. In one embodiment, the measuring the anti-tetanus immune response comprises determining the level of tetanus-specific T cells in the subject. In one embodiment, the measuring the anti-tetanus immune response comprises measuring tetanus-specific T cell proliferation. In one embodiment, the anti-tetanus immune response is measured about 15 days after the administration of the tetanus toxoid and anti-OX40 and/or anti-PD-1 antibody.

In another aspect, the invention features any of the aforementioned embodiments, wherein the anti-PD-1 antibody is replaced by, or combined with, an anti-PD-L1 or anti-PD-L2 antibody.

### VII. Outcomes

With respect to target lesions, responses to therapy may include:

| | |
|---|---|
| Complete Response (CR) | Disappearance of all target lesions. Any |
| (RECIST V1.1) | pathological lymph nodes (whether target |
| | or non-target) must have reduction in short |
| | axis to < 10 mm. |
| Partial Response (PR) | At least a 30% decrease in the sum of the |
| (RECIST V1.1) | diameters of target lesions, taking as |
| | reference the baseline sum diameters. |
| Progressive Disease (PD) | At least a 20% increase in the sum of the |
| (RECIST V1.1) | diameters of target lesions, taking as |
| | reference the smallest sum on study (this |
| | includes the baseline sum if that is the |
| | smallest on study). In addition to the |
| | relative increase of 20%, the sum must also |
| | demonstrate an absolute increase of at least |
| | 5 mm. (Note: the appearance of one or |
| | more new lesions is also considered |
| | progression). |
| Stable Disease (SD) | Neither sufficient shrinkage to qualify for |
| (RECIST V1.1) | PR nor sufficient increase to qualify for |
| | PD, taking as reference the smallest sum |
| | diameters while on study. |
| Immune-related Complete | Disappearance of all target lesions. Any |
| Response (irCR) | pathological lymph nodes (whether target |
| (irRECIST) | or non-target) must have reduction in short |
| | axis to < 10 mm. |
| Immune-related Partial | At least a 30% decrease in the sum of |
| Response (irPR) | diameters of target lesions and all new |
| (irRECIST) | measurable lesions (i.e., Percentage |
| | Change in Tumor Burden), taking as |
| | reference the baseline sum diameters. Note: |
| | the appearance of new measurable lesions |
| | is factored into the overall Tumor Burden, |
| | but does not automatically qualify as |
| | progressive disease until the sum of the |
| | diameters increases by 20% when |
| | compared to nadir. |
| Immune-related | At least a 20% increase in Tumor Burden |
| Progressive Disease (irPD) | (i.e., the sum of diameters of target lesions, |
| (irRECIST) | and any new measurable lesions) taking as |
| | reference the smallest sum on study (this |
| | includes the baseline sum if that is the |
| | smallest on study). In addition to the |
| | relative increase of 20%, the sum must also |
| | demonstrate an absolute increase of at least |
| | 5 mm. Tumor assessments using immune- |
| | related criteria for progressive disease |
| | incorporates the contribution of new |
| | measurable lesions. Each net percentage |
| | change in tumor burden per assessment |
| | accounts for the size and growth kinetics of |
| | both old and new lesions as they appear. |
| Immune-related | Neither sufficient shrinkage to qualify for |
| Stable Disease (irSD) | irPR nor sufficient increase to qualify for |
| (irRECIST) | irPD, taking as reference the smallest sum |
| | diameters while on study. |

With respect to non-target lesions, responses to therapy may include:

| | |
|---|---|
| Complete Response (CR) | Disappearance of all non-target lesions. |
| (RECIST V1.1) | All lymph nodes must be non-pathological |
| | in size (<10 mm short axis). |
| Non-CR/Non-PD | Persistence of one or more non-target |
| (RECIST V1.1) | lesion(s). |
| Progressive Disease (PD) | Unequivocal progression of existing non- |
| (RECIST V1.1) | target lesions. The appearance of one or |
| | more new lesions is also considered |
| | progression. |
| Immune-related | Disappearance of all non-target lesions. All |
| Complete Response (irCR) | lymph nodes must be non-pathological in |
| (irRECIST) | size (<10 mm short axis). |
| Immune-related | Increases in number or size of non-target |
| Progressive Disease (irPD) | lesion(s) does not constitute progressive |
| (irRECIST) | disease unless/until Tumor Burden |
| | increases by 20% (i.e., the sum of the |
| | diameters at nadir of target lesions and any |
| | new measurable lesions increases by the |
| | required amount). Non-target lesions are |
| | not considered in the definition of Stable |
| | Disease and Partial Response. |

Patients treated according to the methods disclosed herein preferably experience improvement in at least one sign of cancer. In one embodiment, improvement is measured by a reduction in the quantity and/or size of measurable tumor lesions. In another embodiment, lesions can be measured on chest x-rays or CT or Mill films. In another embodiment, cytology or histology can be used to evaluate responsiveness to a therapy.

In one embodiment, the patient treated exhibits a complete response (CR), a partial response (PR), stable disease (SD), immune-related complete disease (irCR), immune-related partial response (irPR), or immune-related stable disease (irSD). In another embodiment, the patient treated experiences tumor shrinkage and/or decrease in growth rate, i.e., suppression of tumor growth. In another embodiment, unwanted cell proliferation is reduced or inhibited. In yet another embodiment, one or more of the following can occur: the number of cancer cells can be reduced; tumor size can be reduced; cancer cell infiltration into peripheral organs can be inhibited, retarded, slowed, or stopped; tumor metastasis can be slowed or inhibited; tumor growth can be inhibited; recurrence of tumor can be prevented or delayed; one or more of the symptoms associated with cancer can be relieved to some extent.

In other embodiments, administration of effective amounts of the tetanus toxoid, anti-OX40 antibody and anti-PD-1 antibody according to any of the methods provided herein produces at least one therapeutic effect selected from the group consisting of reduction in size of a tumor, reduction in number of metastatic lesions appearing over time, complete remission, partial remission, or stable disease. In still other embodiments, the methods of treatment produce a comparable clinical benefit rate (CBR=CR+PR+SD≥6 months) better than that achieved by anti-OX40 antibody and anti-PD-1 antibody without a tetanus toxoid. In other embodiments, the improvement of clinical benefit rate is about 20% 20%, 30%, 40%, 50%, 60%, 70%, 80% or more compared to that achieved by an anti-OX40 and anti-PD-1 antibody without a tetanus toxoid.

In other embodiments, administration of effective amounts of the tetanus toxoid and anti-PD-1 antibody according to any of the methods provided herein produces at least one therapeutic effect selected from the group consisting of reduction in size of a tumor, reduction in number of metastatic lesions appearing over time, complete remission, partial remission, or stable disease. In still other embodiments, the methods of treatment produce a comparable clinical benefit rate (CBR=CR+PR+SD≥6 months) better than that achieved by anti-PD-1 antibody without a tetanus toxoid. In other embodiments, the improvement of clinical benefit rate is about 20% 20%, 30%, 40%, 50%, 60%, 70%, 80% or more compared to that achieved by an anti-PD-1 antibody without a tetanus toxoid.

In other embodiments, administration of effective amounts of the anti-OX40 and anti-PD-1 antibodies according to any of the methods provided herein produces at least one therapeutic effect selected from the group consisting of reduction in size of a tumor, reduction in number of metastatic lesions appearing over time, complete remission, partial remission, or stable disease. In still other embodiments, the methods of treatment produce a comparable clinical benefit rate (CBR=CR+PR+SD≥6 months) better than that achieved by an anti-PD-1 antibody without an anti-OX40 antibody. In other embodiments, the improvement of clinical benefit rate is about 20% 20%, 30%, 40%, 50%, 60%, 70%, 80% or more compared to that achieved by an anti-PD-1 antibody without an anti-OX40 antibody. In still other embodiments, the methods of treatment produce a comparable clinical benefit rate (CBR=CR+PR+SD≥6 months) better than that achieved by an anti-OX40 antibody without an anti-PD-1 antibody. In other embodiments, the improvement of clinical benefit rate is about 20% 20%, 30%, 40%, 50%, 60%, 70%, 80% or more compared to that achieved by an anti-OX40 antibody without an anti-PD-1 antibody.

### VIII. Kits and Unit Dosage Forms

Also provided herein are kits which include a pharmaceutical composition containing an anti-OX40 antibody, such as BMS-986178, and/or an anti-PD-1 antibody, such as BMS-936558 or nivolumab, and a pharmaceutically-acceptable carrier, in a therapeutically effective amount adapted for use in the preceding methods. The kits optionally also can include a tetanus toxoid in a therapeutically effective amount adapted for use in the preceding methods. The kits optionally also can include instructions, e.g., comprising administration schedules, to allow a practitioner (e.g., a physician, nurse, or patient) to administer the composition contained therein to administer the composition to a patient having cancer (e.g., a solid tumor). The kit also can include a syringe.

Optionally, the kits include multiple packages of the single-dose pharmaceutical compositions each containing an effective amount of the tetanus toxoid, anti-OX40 antibody or anti-PD-1 antibody for a single administration in accordance with the methods provided above. Instruments or devices necessary for administering the pharmaceutical composition(s) also may be included in the kits. For instance, a kit may provide one or more pre-filled syringes containing an amount of the tetanus toxoid, anti-OX40 antibody or anti-PD-1 antibody.

In one embodiment, the present invention provides a kit for treating cancer or a solid tumor in a human patient, the kit comprising:

(a) a dose of an anti-OX40 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5;

(b) a dose of an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21; and

(c) instructions for using the anti-OX40 antibody and anti-PD-1 antibody in the methods described herein.

In one embodiment, the present invention provides a kit for treating cancer or a solid tumor in a human patient, the kit comprising:

(a) a dose of an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21; and

(b) instructions for using the anti-PD-1 antibody in the methods described herein.

In one embodiment, the present invention provides a kit for treating cancer or a solid tumor in a human patient, the kit comprising:

(a) a dose of a tetanus toxoid;

(b) a dose of an anti-OX40 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5;

(c) a dose of an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21; and

(d) instructions for using the tetanus toxoid, anti-OX40 antibody and anti-PD-1 antibody in the methods described herein.

In one embodiment, the present invention provides a kit for treating cancer or a solid tumor in a human patient, the kit comprising:

(a) a dose of a tetanus toxoid;

(b) a dose of an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21; and

(c) instructions for using the tetanus toxoid, anti-OX40 antibody and anti-PD-1 antibody in the methods described herein.

The following examples are merely illustrative and should not be construed as limiting the scope of this disclosure in any way as many variations and equivalents will become apparent to those skilled in the art upon reading the present disclosure.

The contents of all references, GenBank entries, patents and published patent applications cited throughout this application are expressly incorporated herein by reference.

### EXAMPLES

### Example 1: Dose Regimen Exploration of BMS-986178 in Combination With Nivolumab in Bladder Cancer

BMS-986178 is an anti-OX40 agonist mAb under exploration as a treatment for advanced malignancies. Nivolumab is an anti-programmed cell death-1 (PD-1) monoclonal antibody (mAb) approved for the treatment of metastatic melanoma, non-small cell lung cancer (NSCLC), and advanced renal cell carcinoma (RCC) in multiple countries, and ipilimumab is an anti-cytotoxic T-lymphocyte associated antigen-4 (CTLA-4) mAb approved for the treatment of metastatic melanoma in multiple countries.

OX40 is expressed in several types of human malignancies. Examination of The Cancer Genome Atlas (TCGA) database reveals that OX40 exhibits a broad range of gene expression across these various tumor types. Additionally, correlations were observed between OX40 expression and gene expression signatures associated with specific immune cell infiltrates including CD8+ T-cells, Tregs, and macrophages in multiple tumor types. Those tumor types that revealed the strongest correlation between signatures of immune infiltration and OX40 expression included bladder, cervical, testicular, colorectal, lung, head and neck, and ovarian cancers. Furthermore, in several of these tumor types (including bladder and head and neck cancers), high OX40 expression was associated with prolonged survival.

In order to further understand which tumor types might be most likely to respond to anti-OX40, tumors with the highest levels of OX40 gene expression were selected for further study by immunohistochemistry (IHC) to confirm the presence of OX40 on effector T-cells and Tregs in the tumor indications suggested by the TCGA analysis. OX40+ lymphocytes were present in most samples from all tumor types examined. Among them, OX40+ lymphocytes were present at moderate to high levels in cervical carcinoma (56%), colorectal cancer (CRC) (68%), bladder cancer (BC) (37%), ovarian cancer (OC) (22%), and NSCLC (55%). The presence of tumor infiltrating, OX40+ lymphocytes in these tumor types suggests that they may be likely to respond to treatment with an OX40 agonist antibody.

In addition to this preclinical analysis of OX40 expression on human cancers, others have published correlations between OX40 expression and clinical outcomes in the tumors types identified as candidates for treatment. For example, Weixler et al. recently showed that high OX40 expression on CD8+ T-cells within the tumor microenvironment of CRC patients represents an independent, favorable, prognostic marker in this disease. Weixler et al., Oncotarget 2015;6(35):37588-99.

### Summary of Study Design

This study will be part of a Phase 1/2a, open-label trial of BMS-986178 in subjects with advanced solid tumors that integrates initial BMS-986178 monotherapy with subsequent nivolumab and/or ipilimumab combination therapy. The Phase 1/2a trial has been designed to evaluate the safety profile, tolerability, pharmacokinetics (PK), pharmacodynamics (PD), and preliminary efficacy of BMS-986178 alone or in combination with nivolumab and/or ipilimumab in humans with advanced solid tumors. In addition, the study is expected to identify the recommended Phase 2 dose of BMS-986178 alone or in combination with nivolumab and/or ipilimumab.

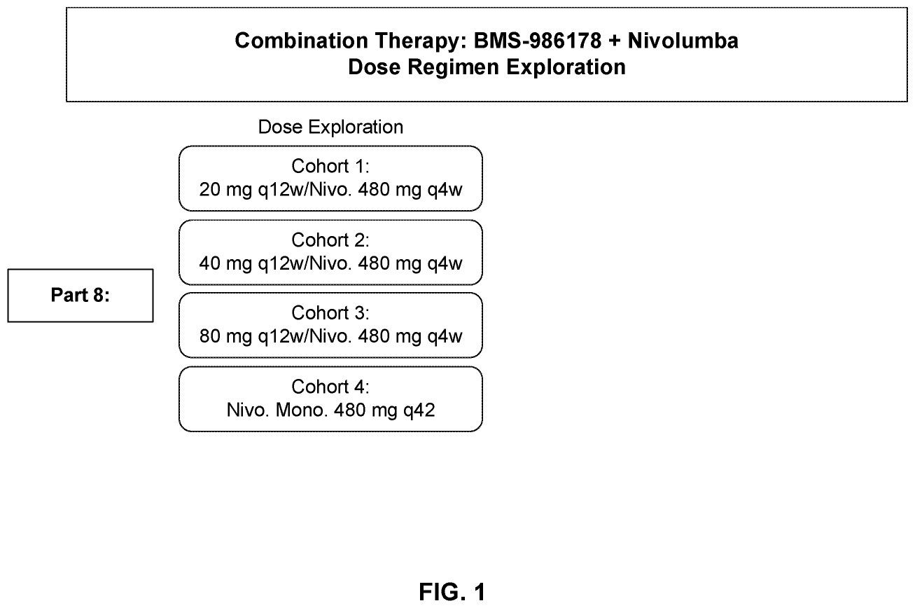

This study is a dose regimen exploration of BMS-986178 in combination with nivolumab or nivolumab monotherapy. Nivolumab will be administered at a flat dose of 480 mg to be administered every 4 weeks. The study design schematic is shown in FIG. 1.

Approximately 20 evaluable subjects with bladder cancer per cohort will be treated.

Cohort 1-3: BMS-986178 will be administered as a flat dose of either 20 mg, 40 mg, or 80 mg q12w in combination with nivolumab flat dose (480 mg; q4w). Each treatment cycle will be 12 weeks in length starting on Day 1 of each cycle. There will be up to 9 cycles, to allow for 24 months of treatment. A tetanus vaccine (Tdap preferred, Td or equivalent after discussion with the medical monitor) will be administered first on Cycle 1 Day 1 prior to administration of nivolumab and BMS-986178.

Cohort 4: Nivolumab monotherapy will be administered as a flat dose of 480 mg (q4w). Each treatment cycle will be 12 weeks in length and will be dosed for up to 9 cycles, 24 months of dosing. Treatment will be given on Day 1, Day 29 and 57 of each cycle. A tetanus vaccine (Tdap preferred, Td or equivalent after discussion with the medical monitor) will be administered first on Cycle 1 Day 1 prior to administration of nivolumab monotherapy.

Administration of a potent recall antigen such as tetanus toxoid primes the immune system, induces an immune response, and promotes a more immunogenic state. The ability of anti-OX40 to enhance a recall response will be determined by monitoring antibodies to tetanus, and proliferative and cytokine responses by CD4+ T-cells after tetanus vaccination. Curti et al., Cancer Res 2013;73:7189-98. Approximately 70% of the general population has protective antibodies to tetanus. Gergen et al., N Engl J Med 1995; 332:761. However, cellular immune responses are usually detectable in the peripheral blood one month after tetanus vaccine. Tetanus has been used as a reporter antigen in cancer patients receiving immunotherapy with vaccines and can be easily monitored. Schuler-Thurner et al., J Immunol 2000; 165: 3492; Curti et al., Cancer Res 2013;73:7189-98. Consequently, tetanus vaccination may provide potent recall response with BMS-986178 in combination with nivolumab or nivolumab monotherapy.

### Investigational Product

BMS-986178, an anti-OX40 agonist monoclonal antibody (mAb) supplied as a sterile 25-mg/mL formulation, is to be administered as an intravenous (IV) infusion alone or in combination with nivolumab per the cohort assignment and the duration of treatment, as indicated in the protocol. Nivolumab, an anti-programmed cell death-1 (PD-1) mAb, is available as a sterile 10-mg/mL formulation to be administered as an IV infusion.

Inclusion Criteria—Target Population: Subjects must be at least 18 years old and have histologic or cytologic confirmation of a malignancy that is advanced (metastatic, recurrent, refractory, and/or unresectable) with measurable disease per RECIST v1.1.

### Bladder Cancer

(i) Histologically or cytologically confirmed urothelial carcinoma (including mixed histologies of urothelial carcinoma with elements of other subtypes) of the renal pelvis, ureter, bladder, or urethra with progression or refractory disease.

(ii) Subjects will need to have a pre-treatment and 2 on-treatment biopsies.

(iii) Prior therapy requirement:

Subjects must have been offered and/or have received or refused 1 prior platinum-based therapy for the treatment of metastatic or locally advanced unresectable disease. Subjects must have not received more than 1 prior systemic therapy. Reason(s) for refusal should be documented.

Subjects must be immunotherapy treatment naive (e.g., no prior therapy with experimental anti-tumor vaccines; any T-cell co-stimulation or checkpoint pathways, such as anti-PD-1, anti-PD-L1, anti-PD-L2, anti-CD137, or anti-CTLA-4 antibody, including ipilimumab; or other medicines specifically targeting T-cells).

(iv) Within 12 months of peri-operative (neo-adjuvant or adjuvant) treatment with a platinum agent in the setting of cystectomy for localized muscle invasive urothelial cancer.

(v) Sequential chemotherapy given as a planned sequence to optimize response will count as 1 regimen.

(vi) Vaccines for infectious disease (e.g., influenza) allowed, provided they are administered ≥2 weeks prior to or ≥2 weeks after study treatment/vaccine.

### Exclusion Criteria—Target Disease Exceptions

a) Subjects with known or suspected CNS metastases or untreated CNS metastases, or with the CNS as the only site of disease, are excluded. However, subjects with controlled brain metastases will be allowed to enroll. Controlled brain metastases are defined as no radiographic progression for at least 4 weeks following radiation and/or surgical treatment (or 4 weeks of observation if no intervention is clinically indicated), and off of steroids for at least 2 weeks, and no new or progressive neurological signs and symptoms.

b) Subjects with carcinomatous meningitis

c) For Bladder Cancer

i) Prior therapy with experimental anti-tumor vaccines, any T-cell co-stimulation or checkpoint pathways, such as anti-PD1, anti-PD-L1, anti-PD-L2, anti-CD137, or anti-CTLA-4 antibody, including ipilimumab, or other medicines specifically targeting T cells is also prohibited in this part of the study.

ii) No prior adverse reaction to tetanus toxoid-containing vaccines.

iii) Subjects with known allergies to egg products, neomycin, or tetanus toxoid are also considered ineligible.

### Summary of Study Periods

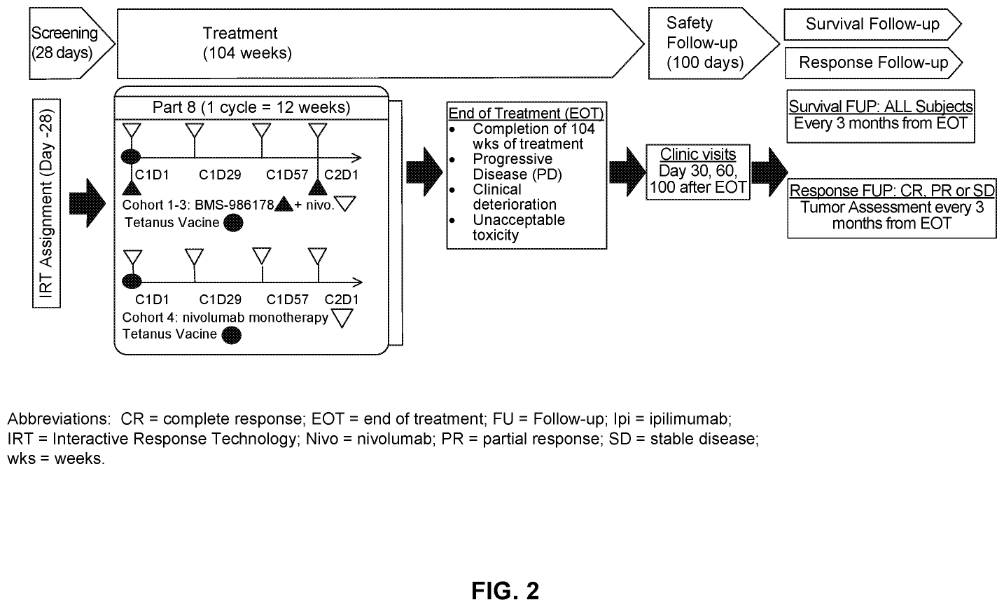

Subjects will complete up to 24 months of dosing, or until meeting protocol-specified discontinuation criteria. Safety Follow-up (approximately 100 days), Response Follow-up, and Survival Long-term Follow-up is up to 2 years from the first dose of study drug. For subjects that have approval for additional cycles up to 2 years of treatment, survival follow-up will be for 6 months after the end of treatment. The study visit schematic is presented in FIG. 2.

### Screening Period

The Screening period will last for up to 28 days. The screening period begins by establishing the subject's initial eligibility and signing of the informed consent form. Subjects will be enrolled using an Interactive Response Technology (IRT).

### Treatment Period

This study will have a treatment period for up to 24 months of dosing. Following each treatment cycle, the decision to treat a subject with the next cycle of study therapy, up to 24 months, will be based on risk/benefit and tumor assessments.

Tumor assessments will be performed every 12 weeks (±1 week). Assessments of partial response (PR) and complete response (CR) must be confirmed at least 4 weeks following initial assessment.

Tumor progression or response endpoints will be assessed using Response Evaluation Criteria In Solid Tumors (RECIST) v1.1.

Subjects with a response of stable disease (SD), PR, or CR at the end of a given cycle will continue to the next treatment cycle. Subjects will generally be allowed to continue study therapy until the first occurrence of one of the following: 1) completion of the maximum number of cycles; 2) progressive disease; 3) clinical deterioration suggesting that no further benefit from treatment is likely; 4) intolerability to therapy; or 5) meeting the criteria for discontinuation of study therapy as outlined in the protocol.

### Safety Follow-Up

Upon completion of study therapy, subjects will enter the Safety Follow-up period. After the end of treatment (EOT) visit, subjects will be evaluated for any new adverse events (AEs) for at least 100 days after the last dose of therapy.

Follow-up visits should occur at Days 30, 60 and 100 after the last dose or the date of discontinuation. Subjects (except those who withdraw consent for study participation) are expected to complete the 3 clinical Safety Follow-up visits regardless of whether they start new anti-cancer therapy.

### Survival Follow-Up

After completion of the Safety Follow-up period, subjects will enter the Survival

Follow-up period. Subjects will be followed approximately every 3 months (12 weeks) until death, lost to follow-up, withdrawal of consent, or conclusion of the study, whichever comes first. The duration of this phase will be for 6 months after the end of treatment.

### Response Follow-Up

After completion of the Safety Follow-up period, all subjects with ongoing SD, PR, or CR at the EOT visit will enter the Response Follow-up period, which will occur simultaneously with the Survival Follow-up period. These subjects will continue to have radiological and clinical tumor assessments every 3 months (12 weeks) during the Response Follow-up period or until disease progression or withdrawal of study consent. Radiological tumor assessments for subjects who have ongoing clinical benefit may continue to be collected after subjects complete the survival phase of the study. Subjects who have disease progression following initial course of study therapy will not be evaluated for response beyond the EOT visit and will be allowed to receive other tumor directed therapy as required.

### Duration of Study

The total duration of study time for any individual subject is expected to be approximately 2.5 years (depending on Part subject is randomized to). The study will end when the last subject completes their last study visit, which is planned to be about 4 years after the start of the study.

Study Population: Subjects must be at least 18 years old and have histologic or cytologic confirmation of a malignancy that is advanced (metastatic, recurrent, refractory and/or unresectable) with measurable disease per RECIST v1.1.

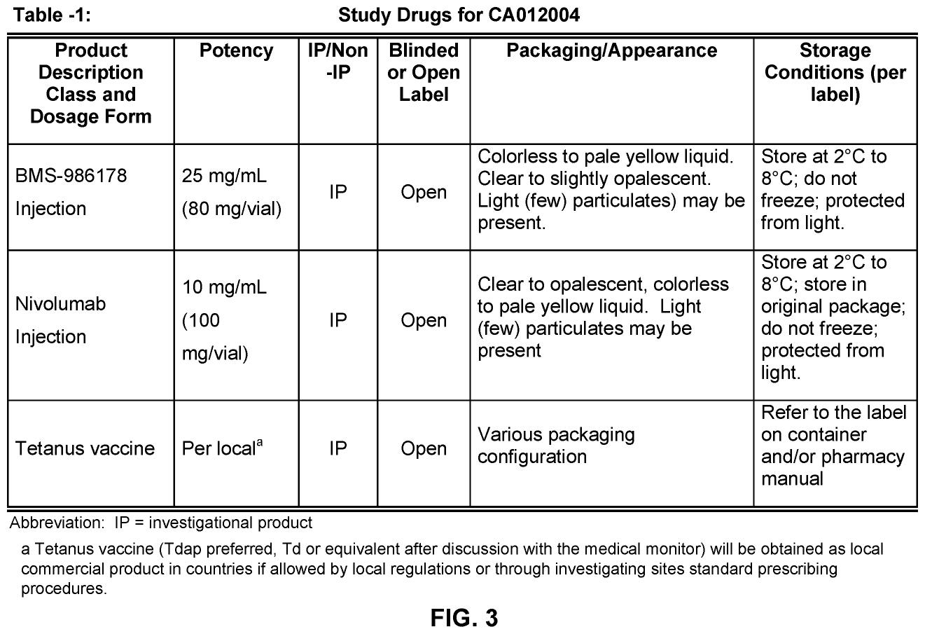

Study Drug: Investigational products are as listed in FIG. 3.

### Study Assessments

Physical examinations, vital sign measurements, 12-lead electrocardiograms (ECGs), and clinical laboratory evaluations will be performed at selected times throughout the dosing interval. Subjects will be closely monitored for AEs throughout the study.
Safety Assessments: AEs will be assessed during the study and for 100 days after the last treatment. AEs will be evaluated according to National Cancer Institute Common Terminology Criteria for Adverse Events v4.03. AEs will be coded using the most current version of Medical Dictionary for Regulatory Activities and reviewed for potential significance and importance. Subjects will be followed until all treatment-related AEs have recovered to baseline or are deemed irreversible by the investigator.
Efficacy Assessments: Disease assessment with computed tomography and/or magnetic resonance imaging as appropriate will be performed at baseline and every 8 weeks (□1 week), then every 12 weeks during the Response Follow-up phases per RECIST v1.1 until discontinuation of treatment or withdrawal from study. Tumor assessments at other time points may be performed if the investigator is concerned about tumor progression. Assessment of tumor response will be reported by the investigator as defined by RECIST v1.1 for subjects with advanced solid tumors.
Pharmacokinetic and Immunogenicity Assessments: Samples for PK and immunogenicity assessments will be collected for subjects receiving BMS-986178 alone or in combination with nivolumab. The PK of BMS-986178 will be characterized by non-compartmental analysis method. Immunogenicity samples will be analyzed for anti-BMS-986178 antibodies and/or anti-nivolumab antibodies by validated immunoassays.
Exploratory Biomarker Assessments: To explore potential predictive markers for clinical response to BMS-986178 in relation to dose and PK, 3 types of specimens will be obtained from all subjects for biomarker testing: (i) whole blood, (ii) serum/plasma, and (iii) tumor tissue.

Sample Size Determination: Approximately 20 evaluable subjects per dose cohort will be treated in Part 8 of the study, BMS-986178 in combination with nivolumab (Cohort 1-3) and monotherapy nivolumab (Cohort 4). Total number of subjects in this Part 8 will be approximately 80.

Efficacy Endpoint: The anti-tumor activity of BMS-986178 alone or in combination with nivolumab and/or ipilimumab will be measured by ORR, duration of response, and progression free survival rate (PFSR) at 24 weeks based on RECIST v1.1. The above will be determined based on tumor measurements occurring at baseline, every 8 weeks (±1 week) during the treatment period, and every 12 weeks during the Survival Follow-up Period.
Best overall response (BOR) is assessed by investigator and/or BICR per RECIST 1.1 criteria.
ORR is defined as the proportion of all treated subjects whose BOR is either CR or PR.
Duration of response, computed for all treated subjects with a BOR of CR or PR, is defined as the time between the date of first response and the date of disease progression or death, whichever occurs first.
PFSR at 24 weeks is defined as the proportion of treated subjects remaining progression-free and surviving at 24 weeks. The proportion will be calculated by the Kaplan-Meier estimate, which takes into account censored data.

Pharmacodynamics Endpoint: Pharmacodynamics will be assessed by the proportion of subjects showing a change in pharmacodynamic biomarkers such as soluble OX40 and peripheral OX40 receptor occupancy along with tumor pharmacodynamic of BMS-986178 in combination with nivolumab or nivolumab monotherapy (Part 8).

Efficacy analyses: The primary efficacy analyses will be performed on all treated subjects. Efficacy analyses based on response-evaluable subjects may be performed for interim analyses when the minimum follow up period is less than sufficient to warrant adequate interpretation of results. Listing of tumor measurements will be provided by subject and study day in each arm and dose level. Individual subject's BOR will be listed based on RECIST 1.1.

To describe the anti-tumor activity of BMS-986178 alone or in combination with nivolumab, ORR will be calculated. ORR and corresponding 2-sided 95% CI by the Clopper-Pearson method will be provided by treatment and/or dose level and tumor type. Median duration of response and corresponding 2-sided 95% CI will be reported by treatment and/or dose level and tumor type. Duration of response will be analyzed using the Kaplan-Meier method.

In addition, PFSR, the probability of a subject remaining progression-free or surviving to 24 weeks, will be estimated by the Kaplan-Meier methodology by treatment, tumor type, and dose level. The corresponding 95% CI will be derived based on Greenwood formula.

OS will be plotted using the Kaplan-Meier method. Median OS and corresponding 2-sided 95% CI will be reported.

### Example 3: Rationale for Pharmacodynamics and Predictive Biomarker Selection

This study is focusing on further optimizing the dose of BMS-986178 in combination with nivolumab. Three dose levels of BMS-986178 and a fixed dose of nivolumab (Cohort 1-3) along with a nivolumab monotherapy (Cohort 4) at a fixed dose are being tested with a tetanus vaccine given on Cycle 1 Day 1 based on prior response and biomarker signals. Therefore, the biomarker selection will include the standard nivolumab assay panel and markers probing for BMS-986178 induced PD biomarkers and functions to assess if BMS-986178 agonist treatment can further enhance nivolumab-driven effects.

Tumor biopsy specimens will be obtained from consenting subjects prior to and during treatment with BMS-986178 in combination with nivolumab and nivolumab monotherapy. On-treatment biopsies at early (D15) and late (D78) sampling time points during the 1st dose of q12w dosing interval will be necessary to accurately assess the immune modulation at different dose-schedule of BMS-986178 in combination with nivolumab or nivolumab monotherapy over time, which allows accurate assessment of the “bell-shaped” response observed for BMS-986178 in combination with nivolumab. The late biopsy sample at D78, prior to the second dosing, could provide better characterization of target engagement for receptor occupancy, in the Q12W regimen, to help understand the duration of pharmacodynamic activity of an agonist as, BMS-986178. Data from pharmacokinetics, peripheral PD such as soluble OX40 and peripheral OX40 receptor occupancy recovery/loss, along with tumor pharmacodynamic data will be used for characterization of immune cell populations, expression of selected tumor markers and relationship between PK, peripheral and intratumoral receptor occupancy to support subsequent dose selection.

SEQUENCE SUMMARY

| SEQ | |
|---|---|
| ID | |
| NO: | SEQUENCE |
| 1 | Heavy Chain Amino Acid Sequence |
| | Anti-OX40 mAb (BMS-986178) |
| | EVQLVQSGGGLVQPGGSLRLSCAGSGFTFSSYAMYWVRQAPGKGLEWVSAIDTDAGTFYA |
| | DSVRGRFTISRDNAKNSLYLQMNSLRAEDTAVYFCARLGEGYFFDYWGQGTLVTVSSAST |
| | KGPSVFPLAPSSKSTSGGTAALGCLVKDYFPEPVTVSWNSGALTSGVHTFPAVLQSSGLY |
| | SLSSVVTVPSSSLGTQTYICNVNHKPSNTKVDKRVEPKSCDKTHTCPPCPAPELLGGPSV |
| | FLFPPKPKDTLMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYTLPPSREEMTK |
| | NQVSLTCLVKGFYPSDIAVEWESNGQPENNYKTTPPVLDSDGSFFLYSKLTVDKSRWQQG |
| | NVFSCSVMHEALHNHYTQKSLSLSPG |
| 2 | Light Chain Amino Acid Sequence |
| | Anti-OX40 mAb (BMS-986178) |
| | EIVLTQSPATLSLSPGERATLSCRASQSVSSYLAWYQQKPGQAPRLLIYDASNRATGIPA |
| | RFSGSGSGTDFTLTISSLEPEDFAVYYCQQRSNWPPTFGGGTKVEIKRTVAAPSVFIFPP |
| | SDEQLKSGTASVVCLLNNFYPREAKVQWKVDNALQSGNSQESVIEQDSKDSTYSLSSTLT |
| | LSKADYEKHKVYACEVTHQGLSSPVTKSFNRGEC |
| 3 | Heavy Chain Variable Region (VH) Amino Acid Sequence |
| | Anti-OX40 mAb (BMS-986178) |
| | EVQLVQSGGGLVQPGGSLRLSCAGSGFTFSSYAMYWVRQAPGKGLEWVSAIDTDAGTFYA |
| | DSVRGRFTISRDNAKNSLYLQMNSLRAEDTAVYFCARLGEGYFFDYWGQGTLVTVSS |
| 4 | Heavy Chain Variable Region (VH) Nucleotide Sequence |
| | Anti-OX40 mAb (BMS-986178) |
| | GAGGTTCAGCTGGTGCAGTCTGGGGGAGGCTTGGTTCAGCCTGGGGGGTCCCTGAGACTC |
| | TCCTGTGCAGGCTCTGGATTCACCTTCAGTAGCTATGCTATGTACTGGGTTCGCCAGGCT |
| | CCAGGAAAAGGTCTGGAGTGGGTATCAGCTATTGATACTGATGCTGGCACATTCTATGCA |
| | GACTCCGTGCGGGGCCGATTCACCATCTCCAGAGACAATGCCAAGAACTCCTTGTATCTT |
| | CAAATGAACAGCCTGAGAGCCGAGGACACGGCTGTGTATTTCTGTGCAAGACTTGGGGAA |
| | GGGTACTTCTTTGACTACTGGGGCCAGGGAACCCTGGTCACCGTCTCCTCA |
| 5 | Light Chain Variable Region (VL) Amino Acid Sequence |
| | Anti-OX40 mAb (BMS-986178) |
| | EIVLTQSPATLSLSPGERATLSCRASQSVSSYLAWYQQKPGQAPRLLIYDASNRATGIPA |
| | RFSGSGSGTDFTLTISSLEPEDFAVYYCQQRSNWPPTFGGGTKVEIK |
| 6 | Light Chain Variable Region (VL) Nucleotide Sequence |
| | Anti-OX40 mAb (BMS-986178) |
| | GAAATTGTGTTGACACAGTCTCCAGCCACCCTGTCTTTGTCTCCAGGGGAAAGAGCCACC |
| | CTCTCCTGCAGGGCCAGTCAGAGTGTTAGCAGCTACTTAGCCTGGTACCAACAGAAACCT |
| | GGCCAGGCTCCCAGGCTCCTCATCTATGATGCATCCAACAGGGCCACTGGCATCCCAGCC |
| | AGGTTCAGTGGCAGTGGGTCTGGGACAGACTTCACTCTCACCATCAGCAGCCTAGAGCCT |
| | GAAGATTTTGCAGTTTATTACTGTCAGCAGCGTAGCAACTGGCCTCCCACTTTCGGCGGA |
| | GGGACCAAGGTGGAGATCAAA |
| 7 | Heavy Chain CDR1 Amino Acid Sequence |
| | Anti-OX40 mAb (BMS-986178) |
| | SYAMY |
| 8 | Heavy Chain CDR2 Amino Acid Sequence |
| | Anti-OX40 mAb (BMS-986178) |
| | AIDTDAGTFYADSVRG |
| 9 | Heavy Chain CDR3 Amino Acid Sequence |
| | Anti-OX40 mAb (BMS-986178) |
| | LGEGYFFDY |
| 10 | Light Chain CDR1 Amino Acid Sequence |
| | Anti-OX40 mAb (BMS-986178) |
| | RASQSVSSYLA |
| 11 | Light Chain CDR2 Amino Acid Sequence |
| | Anti-OX40 mAb (BMS-986178) |
| | DASNRAT |
| 12 | Light Chain CDR3 Amino Acid Sequence |
| | Anti-OX40 mAb (BMS-986178) |
| | QQRSNWPPT |
| 13 | Human OX40 precursor Amino Acid Sequence |
| | MCVGARRLGRGPCAALLLLGLGLSTVTGLHCVGDTYPSNDRCCHECRPGNGMVSRCSRSQ |
| | NTVCRPCGPGFYNDVVSSKPCKPCTWCNLRSGSERKQLCTATQDTVCRCRAGTQPLDSYK |
| | PGVDCAPCPPGHFSPGDNQACKPWTNCTLAGKHTLQPASNSSDAICEDRDPPATQPQETQ |
| | GPPARPITVQPTEAWPRTSQGPSTRPVEVPGGRAVAAILGLGLVLGLLGPLAILLALYLL |
| | RRDQRLPPDAHKPPGGGSFRTPIQEEQADAHSTLAKI |
| 14 | Extracellular domain of mature human OX40 Amino Acid |
| | Sequence |
| | LHCVGDTYPSNDRCCHECRPGNGMVSRCSRSQNTVCRPCGPGFYNDVVSSKPCKPCTWCN |
| | LRSGSERKQLCTATQDTVCRCRAGTQPLDSYKPGVDCAPCPPGHFSPGDNQACKPWTNCT |
| | LAGKHTLQPASNSSDAICEDRDPPATQPQETQGPPARPITVQPTEAWPRTSQGPSTRPVE |
| | VPGGRAVAA |
| 15 | Human OX40 Epitope |
| | DVVSSKPCKPCTWCNLR |
| 16 | human IgG1 constant domain |
| | ASTKGPSVFPLAPSSKSTSGGTAALGCLVKDYFPEPVTVSWNSGALTSGVHTFPAVLQSS |
| | GLYSLSSVVTVPSSSLGTQTYICNVNHKPSNTKVDKRVEPKSCDKTHTCPPCPAPELLGG |
| | PSVFLFPPKPKDTLMISRIPEVICVVVDVSHEDPEVKFNWYVDGVEVHNAKTKPREEQYN |
| | STYRVVSVLTVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSKLTVDKSRW |
| | QQGNVFSCSVMHEALHNHYTQKSLSLSPG |
| 17 | Heavy Chain Amino Acid Sequence |
| | Anti-PD-1 mAb (BMS-936558; 5C4 in WO 2006/121168) |
| | (variable region underlined; constant region bold) |
| | QVQLVESGGGVVQPGRSLRLDCKASGITFSNSGMHWVRQAPGKGLEWVAVIWYDGSKRYY |
| | ADSVKGRFTISRDNSKNTLFLQMNSLRAEDTAVYYCATNDDYWGQGTLVTVSSASTKGPS |
| | VFPLAPCSRSTSESTAALGCLVEDYFPEPVTVSWNSGALTSGVHTFPAVLQSSGLYSLSS |
| | VVTVPSSSLGTKTYTCNVDHKPSNTKVDKRVESKYGPPCPPCPAPEFLGGPSVFLFPPKP |
| | KDTLMISRTPEVTCVVVDVSQEDPEVQFNWYVDGVEVENAKTKPREEQFNSTYRVVSVLT |
| | VLHQDWLNGKEYKCKVSNKGLPSSIEKTISKAKGQPREPQVYTLPPSQEEMTKNQVSLTC |
| | LVKGFYPSDIAVEWESNGQPENNYKTTPPVLDSDGSFFLYSRLTVDKSRWQEGNVFSCSV |
| | MHEALHNHYTQKSLSLSLGK |
| 18 | Light Chain Amino Acid Sequence |
| | Anti-PD-1 mAb (BMS-936558; 5C4 in WO 2006/121168) |
| | (variable region underlined; constant region bold) |
| | EIVLTQSPATLSLSPGERATLSCRASQSVSSYLAWYQQKPGQAPRLLIYDASNRATGIPA |
| | RFSGSGSGTDFTLTISSLEPEDFAVYYCQQSSNWPRTFGQGTKVEIKRTVAAPSVFIFPP |
| | SDEQLKSGTASVVCLLNNFYPREAKVQWKVDNALQSGNSQESVTEQDSKDSTYSLSSTLT |
| | LSKADYEKHKVYACEVTHQGLSSPVTKSFNRGEC |
| 19 | Heavy Chain Variable Region (VH) Amino Acid Sequence |
| | Anti-PD-1 mAb (BMS-936558; 5C4 in WO 2006/121168) |
| | (SEQ ID NO:4 from WO 2006/121168) |
| | QVQLVESGGGVVQPGRSLRLDCKASGITFSNSGMHWVRQAPGKGLEWVAVIWYDGSKRYY |
| | ADSVKGRFTISRDNSKNTLFLQMNSLRAEDTAVYYCATNDDYWGQGTLVTVSS |
| 20 | Heavy Chain Variable Region (VH) Nucleotide Sequence |
| | Anti-PD-1 mAb (BMS-936558; 5C4 in WO 2006/121168) |
| | (SEQ ID NO:60 from WO 2006/121168) |
| | cag gtg cag ctg gtg gag tct ggg gga ggc gtg gtc cag cct ggg |
| | agg tcc ctg aga ctc gac tgt aaa gcg tct gga atc acc ttc agt |
| | aac tct ggc atg cac tgg gtc cgc cag gct cca ggc aag ggg ctg |
| | gag tgg gtg gca gtt att tgg tat gat gga agt aaa aga tac tat |
| | gca gac tcc gtg aag ggc cga ttc acc atc tcc aga gac aat tcc |
| | aag aac acg ctg ttt ctg caa atg aac agc ctg aga gcc gag gac |
| | acg gct gtg tat tac tgt gcg aca aac gac gac tac tgg ggc cag |
| | gga acc ctg gtc acc gtc tcc tca |
| 21 | Light Chain Variable Region (VL) Amino Acid Sequence |
| | Anti-PD-1 mAb (BMS-936558; 5C4 in WO 2006/121168) |
| | (SEQ ID NO:11 from WO 2006/121168) |
| | EIVLTQSPATLSLSPGERATLSCRASQSVSSYLAWYQQKPGQAPRLLIYDASNRATGIPA |
| | RFSGSGSGTDFTLTISSLEPEDFAVYYCQQSSNWPRTFGQGTKVEIK |
| 22 | Light Chain Variable Region (VL) Nucleotide Sequence |
| | Anti-PD-1 mAb (BMS-936558; 5C4 in WO 2006/121168) |
| | (SEQ ID NO:67 from WO 2006/121168) |
| | gaa att gtg ttg aca cag tct cca gcc acc ctg tct ttg tct cca |
| | ggg gaa aga gcc acc ctc tcc tgc agg gcc agt cag agt gtt agt |
| | agt tac tta gcc tgg tac caa cag aaa cct ggc cag gct ccc agg |
| | ctc ctc atc tat gat gca tcc aac agg gcc act ggc atc cca gcc |
| | agg ttc agt ggc agt ggg tct ggg aca gac ttc act ctc acc atc |
| | agc agc cta gag cct gaa gat ttt gca gtt tat tac tgt cag cag |
| | agt agc aac tgg cct cgg acg ttc ggc caa ggg acc aag gtg gaa |
| | atc aaa |
| 23 | Heavy Chain CDR1 Amino Acid Sequence |
| | Anti-PD-1 mAb (BMS-936558; 5C4 in WO 2006/121168) |
| | (SEQ ID NO:18 from WO 2006/121168) |
| | NSGMH |
| 24 | Heavy Chain CDR2 Amino Acid Sequence |
| | Anti-PD-1 mAb (BMS-936558; 5C4 in WO 2006/121168) |
| | (SEQ ID NO:25 from WO 2006/121168) |
| | VIWYDGSKRYYADSVKG |
| 25 | Heavy Chain CDR3 Amino Acid Sequence |
| | Anti-PD-1 mAb (BMS-936558; 5C4 in WO 2006/121168) |
| | (SEQ ID NO:32 from WO 2006/121168) |
| | NDDY |
| 26 | Light Chain CDR1 Amino Acid Sequence |
| | Anti-PD-1 mAb (BMS-936558; 5C4 in WO 2006/121168) |
| | (SEQ ID NO:39 from WO 2006/121168) |
| | RASQSVSSYLA |
| 27 | Light Chain CDR2 Amino Acid Sequence |
| | Anti-PD-1 mAb (BMS-936558; 5C4 in WO 2006/121168) |
| | (SEQ ID NO:46 from WO 2006/121168) |
| | DASNRAT |
| 28 | Light Chain CDR3 Amino Acid Sequence |
| | Anti-PD-1 mAb (BMS-936558; 5C4 in WO 2006/121168) |
| | (SEQ ID NO:53 from WO 2006/121168) |
| | QQSSNWPRT |
| 29 | Complete PD-1 sequence (GenBank Accession No.: AAC51773.1) |
| | MQIPQAPWPVVWAVLQLGWRPGWFLDSPDRPWNPPTFFPALLVVTEGDNATFTCSFSNTS |
| | ESFVLNWYRMSPSNQTDKLAAFPEDRSQPGQDCRFRVTQLPNGRDFHMSVVRARRNDSGT |
| | YLCGAISLAPKAQIKESLRAELRVTERRAEVPTAHPSPSPRPAGQFQTLVVGVVGGLLGS |
| | LVLLVWVLAVICSRAARGTIGARRTGQPLKEDPSAVPVFSVDYGELDFQWREKTPEPPVP |
| | CVPEQTEYATIVFPSGMGTSSPARRGSADGPRSAQPLRPEDGHCSWPL |

What is claimed is:

1. A method of treating cancer or a solid tumor in a human patient, the method comprising
(a) administering to the patient an effective amount of a tetanus toxoid, and
(b) administering to the patient after step (a) an effective amount of each of:
an anti-OX40 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and
(ii) an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21.

2. The method of claim 1, wherein step (b) comprises at least one administration cycle, wherein the cycle is a period of twelve weeks, wherein for each of the at least one cycles, one dose of the anti-OX40 antibody is administered at a dose of 20, 40, or 80 mg and three doses of the anti-PD-1 antibody are administered at a dose of 480 mg.

3. The method of claim 1, wherein step (b) comprises at least one administration cycle, wherein the cycle is a period of twelve weeks, wherein for each of the at least one cycles, one dose of the anti-OX40 antibody is administered at a dose sufficient to achieve about 40% OX40 receptor occupancy and three doses of the anti-PD-1 antibody are administered at a dose of 480 mg.

4. The method of claim 2, wherein the anti-OX40 antibody and anti-PD-1 antibody are administered at the following doses:
(a) 20 mg anti-OX40 antibody and 480 mg of anti-PD-1 antibody;
(b) 40 mg anti-OX40 antibody and 480 mg of anti-PD-1 antibody; or
(c) 80 mg anti-OX40 antibody and 480 mg of anti-PD-1 antibody.

5. The method of any one of claims 1 to 4, wherein the anti-PD-1 and anti-OX40 antibodies are formulated for intravenous administration.

6. The method of any one of claims 1 to 5, wherein the anti-PD-1 and anti-OX40 antibodies are formulated together.

7. The method of any one of claims 1 to 5, wherein the anti-PD-1 and anti-OX40 antibodies are formulated separately.

8. The method of any one of claims 1 to 7, wherein the anti-OX40 antibody is administered prior to administration of the anti-PD-1 antibody.

9. The method of claim 8, wherein the anti-OX40 antibody is administered within about 30 minutes prior to administration of the anti-PD-1 antibody.

10. The method of any one of claims 1 to 7, wherein the anti-OX40 antibody is administered after administration of the anti-PD-1 antibody.

11. The method of any one of claims 1 to 7, wherein the anti-OX40 antibody is administered concurrently with the anti-PD-1 antibody.

12. The method of any one of claims 2 to 11, wherein the treatment consists of up to 9 cycles.

13. The method of any one of claims 2 to 12, wherein the tetanus toxoid is administered on Day 1 of the first cycle.

14. The method of any one of claims 2 to 13, wherein the anti-OX40 antibody is administered on Day 1 of each cycle.

15. The method of any one of claims 2 to 14, wherein the anti-PD-1 antibody is administered on Days 1, 29, and 57 of each cycle.

16. The method of any one of claims 1 to 15, wherein the treatment produces at least one therapeutic effect chosen from a reduction in size of a tumor, reduction in number of metastasic lesions over time, complete response, partial response, and stable disease.

17. The method of any one of claims 1 to 16, wherein the cancer or solid tumor is chosen from bladder, cervical, renal cell, testicular, colorectal, lung, head and neck, and ovarian cancers.

18. The method of claim 17, wherein the cancer or solid tumor is bladder cancer.

19. The method of any one of claims 1 to 18, wherein step (a) comprises the administration of a booster dose of the tetanus toxoid.

20. The method of any one of claims 1 to 18, wherein step (a) comprises the administration of a vaccine.

21. The method of claim 20, wherein the vaccine is Tdap, Td, DT, DTap, or an equivalent thereof.

22. The method of claim 21, wherein the vaccine is Tdap or Td.

23. The method of any one of claims 1 to 22, wherein the anti-OX40 antibody comprises
(a) a heavy chain variable region CDR1 comprising the sequence set forth in SEQ ID NO:7;
(b) a heavy chain variable region CDR2 comprising the sequence set forth in SEQ ID NO:8;
(c) a heavy chain variable region CDR3 comprising the sequence set forth in SEQ ID NO:9;
(d) a light chain variable region CDR1 comprising the sequence set forth in SEQ ID NO:10;
(e) a light chain variable region CDR2 comprising the sequence set forth in SEQ ID NO:11; and
(f) a light chain variable region CDR3 comprising the sequence set forth in SEQ ID NO:12.

24. The method of claim 23, wherein the anti-OX40 antibody comprises heavy and light chain variable regions comprising the sequences set forth in SEQ ID NOs:3 and 5, respectively.

25. The method of claim 24, wherein the anti-OX40 antibody comprises heavy and light chains comprising the sequences set forth in SEQ ID NOs:1 and 2, respectively.

26. The method of any one of claims 1 to 25, wherein the anti-PD-1 antibody comprises
(a) a heavy chain variable region CDR1 comprising the sequence set forth in SEQ ID NO:23;
(b) a heavy chain variable region CDR2 comprising the sequence set forth in SEQ ID NO:24;
(c) a heavy chain variable region CDR3 comprising the sequence set forth in SEQ ID NO:25;
(d) a light chain variable region CDR1 comprising the sequence set forth in SEQ ID NO:26;
(e) a light chain variable region CDR2 comprising the sequence set forth in SEQ ID NO:27; and
(f) a light chain variable region CDR3 comprising the sequence set forth in SEQ ID NO:28.

27. The method of claim 26, wherein the anti-PD-1 antibody comprises heavy and light chain variable regions comprising the sequences set forth in SEQ ID NOs:19 and 21, respectively.

28. The method of claim 27, wherein the anti-PD-1 antibody comprises heavy and light chains comprising the sequences as set forth in SEQ ID NOs:17 and 18, respectively.

29. A kit for treating a cancer or solid tumor in a human patient, the kit comprising:
(a) a dose of an anti-OX40 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5;
(b) a dose of an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21; and
(c) instructions for using the anti-OX40 antibody and anti-PD-1 antibody in the method of any one of claims 1 to 28.

30. An anti-OX40 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, for co-administration to a subject in need thereof with an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21, in at least one cycle, wherein for each cycle one dose of the anti-OX40 antibody is administered at a dose of 20, 40, or 80 mg and three doses of the anti-PD-1 antibody are administered at a dose of 480 mg, and wherein an effective amount of a tetanus toxoid is administered before the administration of the anti-OX40 and anti-PD-1 antibodies.

31. A method of treating cancer or a solid tumor in a human patient, the method comprising administering to the patient an effective amount of each of:
(a) an anti-OX40 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and
(b) an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21, and
wherein the method comprises at least one administration cycle, wherein the cycle is a period of twelve weeks, wherein for each of the at least one cycles, one dose of the anti-OX40 antibody is administered at a dose of 20, 40, or 80 mg and three doses of the anti-PD-1 antibody are administered at a dose of 480 mg.

32. A method of treating a solid tumor in a human patient, the method comprising administering to the patient an effective amount of each of:
(a) an anti-OX40 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and
(b) an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21, and
wherein the method comprises at least one administration cycle, wherein the cycle is a period of twelve weeks, wherein for each of the at least one cycles, one dose of the anti-OX40 antibody is administered at a dose sufficient to achieve about 40% OX40 receptor occupancy and three doses of the anti-PD-1 antibody are administered at a dose of 480 mg.

33. A kit for treating cancer or a solid tumor in a human patient, the kit comprising:
(a) a dose of an anti-OX40 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5;
(b) a dose of an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21; and
(c) instructions for using the anti-OX40 antibody and anti-PD-1 antibody in the method of claim 31 or claim 32.

34. An anti-OX40 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, for co-administration to a subject in need thereof with an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21, in at least one cycle, wherein for each cycle one dose of the anti-OX40 antibody is administered at a dose of 20, 40, or 80 mg and three doses of the anti-PD-1 antibody are administered at a dose of 480 mg.

35. A method of treating cancer or a solid tumor in a human patient, the method comprising
(a) administering to the patient an effective amount of a tetanus toxoid, and
(b) administering to the patient after step (a) an effective amount of an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21.

36. The method of claim 35, wherein step (b) comprises at least one administration cycle, wherein the cycle is a period of twelve weeks, wherein for each of the at least one cycles, three doses of the anti-PD-1 antibody are administered at a dose of 480 mg.

37. A kit for treating cancer or a solid tumor in a human patient, the kit comprising:
(a) a dose of an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21; and
(b) instructions for using the anti-PD-1 antibody in the method of claim 35 or claim 36.

38. An anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21, for co-administration to a subject in need thereof with an effective amount of a tetanus toxoid, in at least one cycle, wherein for each cycle three doses of the anti-PD-1 antibody are administered at a dose of 480 mg, and wherein the tetanus toxoid is administered before the administration of the anti-PD-1 antibody.

39. A method of treating a solid tumor in a human patient, the method comprising administering to the patient an effective amount of an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21,
wherein the method comprises at least one administration cycle, wherein the cycle is a period of twelve weeks, wherein for each of the at least one cycles, three doses of the anti-PD-1 antibody are administered at a dose of 480 mg.

40. A kit for treating a solid tumor in a human patient, the kit comprising:
(a) a dose of an anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21; and
(b) instructions for using the anti-PD-1 antibody in the method of claim 39.

41. An anti-PD-1 antibody comprising CDR1, CDR2 and CDR3 domains of the heavy chain variable region having the sequence set forth in SEQ ID NO:19, and CDR1, CDR2 and CDR3 domains of the light chain variable region having the sequence set forth in SEQ ID NO:21, for administration to a subject in need thereof in at least one cycle, wherein for each cycle three doses of the anti-PD-1 antibody are administered at a dose of 480 mg.
